(19) United States
(12) Patent Application Publication
(10) Pub. No.: US 2010/0112072 A1
(43) Pub. Date: May 6, 2010

(54) Controllable Virus/Protein Assemblies and Methods of Making the Same

(75) Inventors: Qian Wang, Columbia, SC (US); Tao Li, Columbia, SC (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/539,324

(22) Filed: Aug. 11, 2009

Related U.S. Application Data
(60) Provisional application No. 61/188,579, filed on Aug. 11, 2008.

(51) Int. Cl. A61K 9/14; C12N 7/00; C08L 89/00; A61K 47/32; C08F 8/00; A61P 43/00

(52) U.S. Cl. 424/490; 435/235.1; 525/54.1; 514/772.1; 525/55; 977/773

(57) ABSTRACT

Methods of forming a bionanocomposite defining a shell and a core are generally provided along with the bionanocomposites themselves. The method includes non-covalently attaching biomacromolecules about a polymeric core such that the biomacromolecules cover at least about at least about 50% of the surface area of the polymeric core to form a shell. The polymeric core includes a polymer having pyridine functional groups. In one particular embodiment, the biomacromolecules can be attached to the polymeric core by combining an organic solution containing the polymer in an organic solvent with an aqueous solution containing the biomacromolecules to form an emulsion, mixing the emulsion, and removing the organic solvent.

### PRIORITY INFORMATION

The present application claims priority to U.S. Provisional Patent Application Ser, No. 61/188,579 filed on Aug. 11, 2008 entitled “Novel Material Development by Controllable Virus/Protein Assembly”, the disclosure of which is incorporated by reference herein.

### GOVERNMENT SUPPORT CLAUSE

The present invention was developed with funding from the National Science Foundation under award CHE-0748690 and Department of Defense award W911NF-04-1-019. Therefore, the government retains certain rights in this invention.

### BACKGROUND

Proteins and bionanoparticles are the key building blocks of all biological matters. The study of their self-assembly behavior with various polymers is of increasing interest for development of functional materials. Compared to inorganic or organic nanoparticles, bionanoparticles are truly monodisperse and can be modified in a well defined manner. Both of these attributes are critical for the quantitative understanding and control the self-assembly structure.

Bionanoparticles are ideal templates and scaffolds for generating nano-based materials with hierarchical structures as they are highly ordered, their detailed structures are known, and they have the ability to be modified both chemically and genetically. The assembly of bionanoparticles will lead to inside understanding of assembly process of biomacromolecules, which can be applied in the assembly of other protein or protein complexes. In particular, protein assemblies have potential applications in drug and protein deliveries.

Hierarchical assemblies of nanoparticles to form core-shell nanostructures have potential applications ranging from drug delivery to photovoltaics, catalysis and optics. Inorganic nanoparticles, such as gold nanoparticles and silica nanoparticles, are the most often used starting materials to form such kind of core-shell structures. For most biomedical applications, it is necessary to use biomacromolecules impart biological functionality and biocompatibility.

It remains a great interest to incorporate biomacromolecules, like proteins and bionanoparticles (BNPs), into core-shell nano-assemblies due to their fragility, BNPs, such as viruses and virus-like biogenic assemblies, are promising building blocks for materials development since they are monodisperse in size and shape, and can be functionalized in a robust, well-defined manner.

As such, a need exists for a simple method of forming bionanoparticles that retain the functionality of the biomolecules.

### SUMMARY

Objects and advantages of the invention will be set forth in part in the following description, or may be obvious from the description, or may be learned through practice of the invention.

The present disclosure is generally directed toward methods of forming a bionanocomposite defining a shell and a core and the bionanocomposites themselves. The method includes non-covalently attaching biomacromolecules about a polymeric core such that the biomacromolecules cover at least about at least about 50% of the surface area of the polymeric core to form a shell. The polymeric core includes a polymer having pyridine functional groups. In one particular embodiment, the biomacromolecules can be attached to the polymeric core by combining an organic solution containing the polymer in an organic solvent with an aqueous solution containing the biomacromolecules to form an emulsion, mixing the emulsion, and removing the organic solvent.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1 shows an illustration of the formation of VP-co-Polymer nanocomposites via (a) surface polymerization and (b) co-assembly methods.

FIG. 1a shows a schematic representation of the formation of biomaterials composed of proteins and P4VP. Step a, b show the formation of aggregations (A) and colloids (C), respectively.

FIG. 2 shows an illustration of the formation of CPMV-co-P4VP raspberry-like nanocomposites via non-covalent interactions.

FIG. 3 shows (a) a dynamic light scattering diagram, and representative TEM images (c, d) and SEM images (e, f) of CPMV-co-P4VP composites (the mass ratio M_{CPMV}/M_{P4VP} is 0.055) after final dialysis, showing the viral particles on the surface. The areas framed using rectangles are enlarged in (c) and (e) accordingly. TEM image of a pure P4VP particle after dialysis is shown in (b), which has very smooth surface, All scale bars stand for 100 nm.

FIG. 4 shows representative FESEM images (a, c) and TEM images (b, d) of CPMV-co-P4VP (the mass ratio MCPMV/MP4VP is 0.11) in water/DMF (v/v=33:1) (for a and b) and in pure water after dialysis (c and d). All scale bars stand for 100 nm.

FIG. 5 shows DLS analysis of CPMV-co-P4VP composites with different weight ratios.

FIG. 6 shows TEM (a-b) and FESEM (c-e) images of Ferritin-co-P4VP. All scare bars in b-e are 50 nm.

FIG. 7 shows (a) a scheme of TMV-co-P4VP and (b-d) FESEM images of TMV-co-P4VP.

FIG. 8 shows TEM images of (a-b) CPMV-co-P2VP and (c-d) CPMV-co-PS-b-P4VP composites.

FIG. 9 shows TEM images of CPMV-co-P4VP composites at different pH values of 3.7, 5.3, 6.5, 7.3, 8.8.

FIG. 10 shows fluorescent spectra of pyrene in four different systems.

FIG. 11 shows DOX drug uptake cure and its release at different pH.

FIG. 12 shows a schematic illustration of the formation of TYMV-co-P4VP raspberry-like colloids.

FIG. 13 shows TEM (a, b), DLS (c), and FESEM (d) analysis of TYMV-co-P4VP. The mass ratio of TYMV and P4VP is 0.2. All scale bars are 100 nm.

FIG. 14 shows DLS analysis of TYMV-co-P4VP with different M_{TYMV}/M_{P44VP}: (a) 0.3, (b) 0.2 and (c) 0.1. Inset is the data from DLS analysis and theoretical calculation.

FIG. 15 shows (a-c) representative FESEM images of TYMV-co-P4VP with different sizes, and (d, e) Schematic model of the TYMV parking on P4VP. All scale bars are 100 nm.

FIG. 16 shows an illustration of the formation of TMV-co-P4VP core-shell like nanocomposites via non-covalent interactions.

FIG. 17 shows representative TEM images (a-c) of TMV-co-P4VP-A-C composites (the mass ratio M_{TMV}/M_{P4VP} is 0.2) and (d) intermediate of TMV-co-P4VP. Inset is the FESEM images of each state. All TEM scale bars stand for 500 nm and FESEM scale bars are 100 nm.

FIG. 18 shows typical TEM images (a-c) and (d) of TMV-co-P4VP (the mass ratio M_{CPMV}/M_{P4VP} is 0.2) in pure water after dialysis at different times 0 d, 3d, 7d and in water/DMF (v/v=33:1) (for d) All scale bars stand for 500 nm.

FIG. 19 shows DLS analysis of TMV-co-P4VP composites with different weight ratios.

FIG. 20 shows representative FESEM images (a-b) for small samples and (c-e) for large samples. TMV-co-P4VP-C (the mass ratio M_{TMV}/M_{P4VP} is 0.20) in pure water. All scale bars stand for 100 nm.

FIG. 21 shows TEM images (a) P4VP-b-PS-b-P4VP triblock copolymer micelles (b) TMV induced micelles of TMV-co-P4VP-b-PS-b-P4VP copolymer micelles (d) P4VP-b-PS-b-P4VP-co-TMV micelles. FESEM images of TMV induced micelles of P4VP-b-PS-b-P4VP-co-TMV copolymer micelles.

FIG. 22 shows TEM images: (a) P4VP-PS-P4VP triblock copolymer micelles with TMV in dioxane; (b) P4VP-PS-P4VP copolymer micelles in dioxane; (c) P4VP-PS-P4VP triblock copolymer micelles with TMV in DMF; and (d) P4VP-PS-P4VP copolymer micelles in DMF.

FIG. 23 shows a schematic representation of TMV complexed with cyclodextran (CD) to form the long fiber, then can be interact with PEG to form the hydrogel.

FIG. 24 shows TEM image (a), SEM image (b), fluorescence microscopy image (c), and dark field image of rhodamine modified M13 (d).

FIG. 25 shows TEM images of different Protein-co-P4VP micelles (a) BSA-co-P4VP,(b) ChT-co-P4VP, (c) HSA-co-P4VP, (d) Hem-co-P4VP, (e) Lys-co-P4VP, and (f) Myo-co-P4VP.

FIG. 26 shows an illustration of the formation of CPMV/P4VP core-shell like nanocomposites via surface polymerization.

FIG. 27 shows TEM images of (a) P4VP, (b) CPMV-P4VP, and (c-d) P4VP-CPMV.

FIG. 28 shows an illustration to form TMV-Br initiator and TMV-Poly(HEMA).

FIG. 29 shows (a) Optical image of TMV-Poly(HEMA), (b) sucros-gradient image of TMV-Br macro-initiator and TMV-PolyHEMA, (c,d) TEM image of TMV-Br and TMV-Poly(HEMA).

FIG. 30 is a phase diagram showing the transitions in the proteins-P4VP structures. A: aggregations of proteins-P4VP; B: hydrophilic P4VP; C: colloids of proteins-P4VP; and D: denatured proteins.

FIG. 31 shows circular dichroism of ChT and ChT-P4VP (1-3) samples (top), BSA and BSA-P4VP (1-3) samples (middle), and BSA and BSA-PCL-b-P2VP samples (bottom).

FIG. 32 shows bioactivity assays of ChT and BSA whereby 1, 2, and 3 correspond to the protein-P4VP 1, 2, and 3, respectively.

FIG. 33 shows (a) TEM image of the Apo E4-P4VP conjugated with NHS-fluorescein, (b) fluorescent confocal images of human umbilical-vein endothelial cells after being treated for 24 h with Apo E4-P4VP conjugated with NHS-fluorescein. (c) and (d) are the enlarged confocal images. Scale bars in (a) and (b-d) are 200 nm and 20 μm, respectively

Repeat use of reference characters in the present specification and drawings is intended to represent the same or analogous features or elements of the present invention.

### DEFINITIONS

As used herein, the term “biomacromolecule” means a material having or promoting a biological activity and has a size larger than an oligomer (e.g., greater than about 10 monomer units). Biomacromolecules include bionanoparticles (such as virus particles and virus-like biogenic assemblies), proteins, enzyme complexes, etc.

As used herein, the prefix “nano” refers to the nanometer scale (i.e., from about 1 nm to about 999 nm). For example, particles having an average diameter on nanometer scale (i.e., from about 1 nm to about 999 nm) are referred to as “nanoparticles”. Particles having a size of greater than 1,000 nm (i.e., 1 μm) are generally referred to as “microparticles”, since the micrometer scale generally involves those particles having an average diameter of greater than 1 μm.

As used herein, the term “polymer” generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

The term “protein” is defined to include any molecular chain of amino acids that is capable of interacting structurally, enzymatically or otherwise with other proteins, polypeptides or any other organic or inorganic molecule.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

Generally speaking, core-shell assemblies of virus-polymer bionanocomposites based on the non-covalent interaction between the polymers and biomacromolecules is described in the present disclosure. Because the bionanocomposites are held together via non-covalent bonding (e.g., van der Waals forces, hydrogen bonding, hydrophobic-hydrophilic interactions and other electrostatic interactions), the formation of the bionanocomposites can be readily reversed, releasing the biomacromolecules from the surface of the bionanocomposite and any other material from within the bionanocomposite. Additionally, the tertiary structures and the biological properties of the biomacromolecules can be retained during the process.

The core-shell bionanocomposites can generally be described as having a polymeric core with biomacromolecules attached about the outer surface of the core to form a shell. These core-shell bionanocomposites can be relatively stable and can maintain the original morphology for over one month at room temperature. These bionanoparticles can also represent known self-assembled architecture mono-dispersed at the nanometer level (e.g., having a diameter from about 10 nm to about 200 nm). Their composition can be controlled by molecular biology techniques, and they can be easily made inexpensively on the gram to kilogram scale. Their three dimensional structure can be characterized with atomic resolution.

In addition to being reversible, the assembly processes can be controlled by varying the solution pH values. Therefore, the core/shell bionanocomposites may have potential applications in drug delivery and controlled release induced by a pH change. For example, the bionanocomposite can be stable in an aqueous solution when the pH is above about 5, such as when the pH is above about 4. However, when the pH of the aqueous solution containing the bionanocomposite is lowered, the bionanocomposite can disassociate due to reduced interaction strength between the polymeric core and the biomacromolecules in the shell. Without wishing to be bound by theory, it is believed that the introduction of excess hydrogen protons (H⁺) in the solution interferes with the non-covalent bonding between the shell and the core materials. For example, the shell and core materials of the bionanocomposite can disassociate in an aqueous solution having a pH below about 5, such as below about 4. In one particular embodiment, the shell and core materials of the bionanocomposite can disassociate in an aqueous solution having a pH below about 3, such as from a pH of about 1 to about 3.

### I. Methods of Forming the Core-Shell Nanostructures

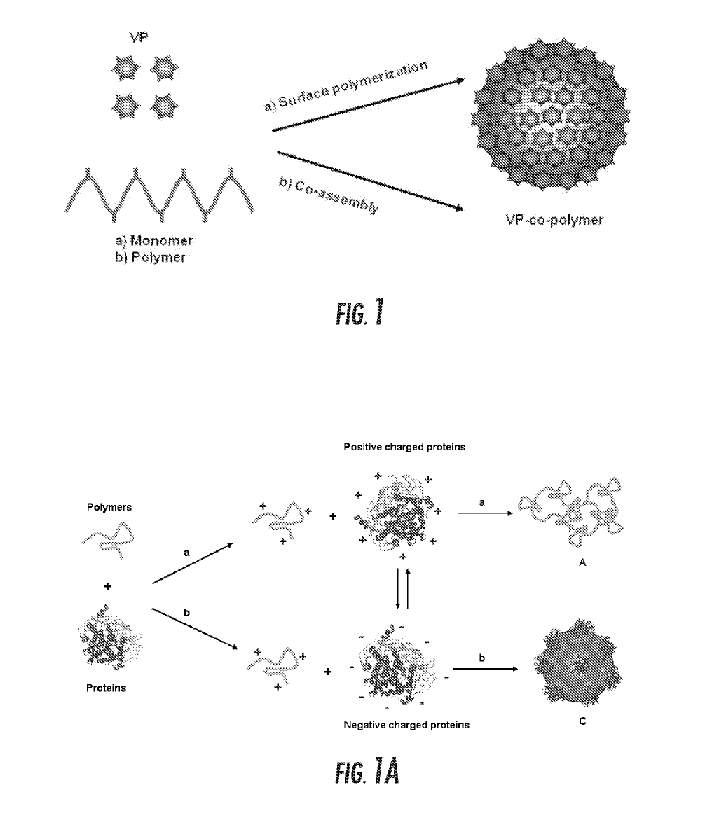

As shown in FIG. 1, for example, there are two general approaches to form these core-shell nanostructures.

The first method is directed to preparing the bionanocomposites based on the self-assembly along with polymerization including emulsion and living polymerization.

The second method is directed to forming composites based on the controlled self-assemblies using the biomacromolecules as the building blocks. According to this method, the outer surface of a polymer particle is surrounded with the biomacromolecules to form the shell of the bionanocomposite. This method is discussed in greater detail below.

The method involves combining a polymer (or monomers forming a polymer) in an organic solvent (e.g., ethanol, dimethylformamide (DMF), etc., and mixtures thereof) with biomacromolecules in an aqueous solution to form an emulsion. Mixing of the two solutions can be performed according to any method. In one particular embodiment, the organic solution containing the polymer can be slowly added (e.g., dripped) into the aqueous solution while mixing. After mixing, the organic solvent can be removed (e.g., through dialysis, evaporation, etc.). In one embodiment, the organic solution containing the polymer can be slowly added (e.g., dripped) into a heated aqueous solution, causing the organic solvent to evaporate and leaving the polymer in the aqueous solution. The removal of the organic solvent results in the polymer chains aggregating into polymeric particles, due to their insolubility in the remaining aqueous solution, forming the polymeric core. Then, the biomacromolecules can be attached to the outer surface of the particle core to form the core-shell bionanocomposite.

No matter the method of forming the bionanocomposites, the resulting core-shell bionanocomposites can have good coverage of the biomacromolecules about the outer surface of polymeric core (e.g., covering at least about 50% of the surface area of the polymeric core, such as from about 60% to about 95%). Since bionanoparticles (e.g., viruses and virus-like particles), proteins (e.g., ferritins, heat shock protein cages, etc.), and enzyme complexes are highly organized scaffolds with robust chemical and physical properties and fascinating structural symmetries, myriad BNPs have drawn great attention in the past decade in functional materials development. These methods can allow the synthesis of hierarchically assembled composite colloids using BNPs as building blocks, which can lead to broad potential applications including drug delivery and tissue engineering. In certain embodiments, the polymer core can contain another material for delivery within the core-shell bionanoparticle.

A pH adjuster (e.g., an acid or a base) and/or buffer may also be included in the aqueous solution to control the association of the shell and core materials.

### II. Viral Particles and Proteins

Several types of biomacromolecules (e.g., viral particles and/or proteins) can be used to assemble the core-shell bionanocomposites. As stated, these biomacromolecules can form the shell of the core-shell biocomposites and can be, for example, used to as a scaffold for bioengineering. Several types of viral particles can be used as the biomacromolecules to form the shell of the bionanocomposite, including but not limited to, cowpea mosaic virus (CPMV), turnip mosaic virus (TYMV), tobacco mosaic virus (TMV), bacteriophage M13, and bacteriophage P22 etc., and combinations thereof.

Cowpea mosaic virus (CPMV) is a spherical particle, measuring about 29 nm in diameter. CPMV can be simply isolated from infected plants in yields of 1-2 grams per kg of leaves as known in the art. Sixty copies of the two-protein asymmetric unit are assembled in an icosahedral pattern around the single-stranded viral genomic RNA to form the virus particle. The chemistry of CPMV has been studied extensively, and it is possible to make the insertion of exogenous peptides or the mutation of existing residues for the purposes of engineering novel functions.

TYMV is one of the best known of the small RNA viruses. It is the type member of the tymovirus group, a nonenveloped plant virus made of a positive single stranded RNA. It shows T=3 icosahedral symmetry and is made of 180 chemically identical protein subunits of 20,000 Da. Large quantities of TYMV can be isolated from infected turnips or Chinese cabbage leafs. TYMV is stable from 4° C. to RT indefinitely and 60° C. for several hours. It is also stable to a wide pH range (4-10) and up to 50% organic solvent. TYMV was one of the first isometric viruses to be studied by x-ray crystallographic analysis and by electron microscropy using negative staining. The capsid has of 32 knob-like structures, and each of these knobs correspond to 20 hexamers and 12 pentamers of the coat protein arranged icosahedrally. The chemistry of TYMV has been extensively studied.

TMV is one of the simplest viruses known. Each viral particle has 2130 identical protein subunits arranged in a helical motif around a single stand of RNA to produce a hollow protein tube. The internal and external surfaces of the protein have repeated patterns of charged amino acid residues, such as glutamate, aspartate, arginine, and lysine. The rod like TMV is 300 nm in length and 18 nm in diameter, The chemistry of TMV has been studied extensively. It has been previously demonstrated that non-covalent interactions can promote the polymerization of anilines exclusively on the surface of tobacco mosaic virus to form the conductive nanowires. TMV based materials also find great potential applications in nanoelectronics and energy harvesting devices.

Proteins can also form the shell of the core-shell nanoparticles through co-assembly with the functionalized polymers (e.g., P4VP and other biodegradable copolymers such as poly(c-caprolactone)-block-poly(2-vinyl pyridine) (PCL-b-P2VP), and still retain their bioactivities. Exemplary proteins suitable for use in the bionanocomposite shell include but are not limited to pepsin (Pep) (pI 2.8, M_{w} 35.0 kDa), bovine serum albumin (BSA) (pI 4.8, M_{w} 66.3 kDa), avidin (Avd) (pI 10.5, M_{w} 69.0 kDa), lysozyme (Lys) (pI 11.0, M_{w} 14.4 kDa), papain (Pap) (pI 9.6, M_{w} 23.0 kDa), ovabumin (Ova) (pI 5.1, M_{w} 45.0 kDa), cytochrome c (Cyt) (pI 10.3, M_{w} 12.0 kDa), concanavalin A (Con A) (pI 4.5, M_{w} 104.0 kDa), trpsin (Trp) (pI 10.5, M_{w} 23.3 kDa), ribonuclease A (Rib A) (pI 9.4, Mw 13.7 kDa), α-chymotrypsin (ChT) (pI 8.5, Mw 25.0 kDa), hemoglobin (Hem) (pI 6.8, Mw 64.5 kDa), ferritin (Fer) (pI 4.5, Mw 750.0 kDa), human serum albumin (HSA) (pI 5.2, Mw 69.4 kDa), streptavidin (Str) (pI 5.0, M_{w} 53.0 kDa), porcine stomach mucin (Psm) (pI 4.4, M_{w} 103.0 kDa), lipase (Lip) (pI 5.6, M_{w} 58.0 kDa), geltin type B (Gel B) (pI 4.8, M_{w} 60.0 kDa), horseradish peroxidase (HRP) (pI 7.2, M_{w} 40.0 kDa), and apolipoprotein E4 (Apo E4) (pI 5.5, M_{w} 59.0 kDa).

Of particle advantage, proteins with isoelectric points (pIs) higher than 9 can be protonized at pH 7.8 to form a positive charge. The isoelectric point (pI) is the pH at which a particular molecule or surface carries no net electrical charge. In the case of proteins, the isoelectric point mostly depends on seven charged amino acids: glutamate (δ-carboxyl group), aspartate (R-carboxyl group), cysteine (thiol group), tyrosine (phenol group), histidine (imidazole side chains), lysine (E-ammonium group) and arginine (guanidinium group). Additionally, the charge of protein terminal groups (NH₂ and COOH) is taken into account. Each of them has its unique acid dissociation constant referred to as pK. The net charge of the protein is in tight relation with the solution (buffer) pH. Then, the Henderson-Hasselbach equation can be used to calculate protein charge in certain pH

The mechanism is proposed for the formation of A and C. Driven by the interaction between the pyridine groups of P4VP and the carboxyl groups lying on the periphery of the proteins, as the P4VP ethanol solution was dropped into protein solution, P4VP could be protonated and carry a positive charge. Proteins with pIs higher than 9 carry a net positive charge at pH 7.8. However, upon the addition of P4VP, the carboxyl groups on these proteins are deprotonated; thus, the net charge of the protein becomes less positive. The electrostatic interactions between both positive charged P4VP and proteins repelled each other, thereby P4VP formed A, as shown step a in FIG. 1a. However, proteins with pI lower than 7.8 carried the negative charge. After the addition of P4VP, the carboxyl groups on proteins are deprotonated. As a result, the net charge of the proteins becomes more negative. The interaction between positive charge of P4VP and negative charge proteins results in the formation of C, as shown step b in FIG. 1a. ChT with pI 8.5 is closer to pH of 7.8, as P4VP protonated, ChT might transfer from a positive charge to a negative charge, thereby the C was formed. Therefore, the formation of C and A are mainly based on the electrastatic interactions between P4VP and proteins.

Ferritin proteins can also be used to form the shell. Ferritins are a family of iron storage protein spheres found mainly in liver and spleen, which have attracted many research interests due to their facinating structural features and biological properties. Ferritin that is devoid of iron core provides a cage-like structure (often referred to as apoferritin). For example, a horse spleen apoferritin (apo-HSF) cage contains 24 structurally equivalent subunits arranged by 432 symmetry into a hollow, roughly spherical shell of inner diameter 8 nm and outer diameter 12.5 nm.

Compared to the template synthesis method, the presently disclosed system has its unique advantages: (i) the structures can be easily formed based on the co-assembly of polymers and proteins; (ii) size-controlled nanoparticle-proteins structures can be readily obtained by changing the mass ratios of polymers and proteins; (iii) proteins can still retain their functionalities, especially for some antibodies such as apolipoprotein, which can be used as the potential drug delivery vehicles.

### III. Polymers

The polymer material used to co-assemble with BNPs and other proteins to form core-shell structures can be sufficiently configured to interact with the viral particles and/or proteins. For example, the polymer can include a charged functional group extending from the polymeric backbone. In one embodiment, the charged function group can be an amine group (e.g., a primary, secondary, tertiary, or cyclic amine group).

In one particular embodiment, the polymer can be functionalized with a plurality of pyridine functional groups. A pyridine group is an aromatic heterocyclic organic compound with the general chemical formula R—C₅H₄N in an unprotonated state. Not only do the pyridine functional groups provide a sufficient charge, the degree of attraction between the pyridine group of the polymer and the viral particle and/or protein can also be controlled by varying the pH of the solution. In one particular embodiment, the assembly processes can take place at pH values ranging from about 5 to about 8, while the pyridine units maintain the unprotonated form. However, at lower pH ranges (e.g., from about 3 to about 4) the pyridine group tends to protonate and the core/shell structures can dissociate and bioparticles can be recovered.

Suitable polymers having pyridine functional groups extending from the polymer backbone include, but are not limited to, poly(4-vinylpyridine) (P4VP), poly(2-vinylpyridine) (P2VP), and copolymers of P4VP or P2VP such as poly(styrene-b-4-vinyl pyridine) (PS-b-P4VP), poly(styrene-b-2-vinyl pyridine) (PS-b-P2VP), poly(2-vinyl pyridine-b-ε-caprolactone) (P2VP-b-PCL), polyethylene oxide-b-4-vinyl pyridine) (PEO-b-P4VP), poly(styrene-b-4-vinylpyridine-b-styrene)(PS-b-P4VP-b-PS), poly(4-vinyl pyridine-b-styrene-b-4-vinyl pyridine) (P4VP-b-PS-b-P4VP), poly(styrene-b-4-vinyl pyridine-b-ethylene oxide) (PS-b-P4VP-b-PEO). Since P4VP and P2VP (and copolymers thereof) have both hydrophobic and hydrophilic motifs, the morphological transformation of the polymer can be controlled by solvent composition and pH. The other polymer such as poly(3-(2-methoxyethoxy)methythiophenes) (PMT), polyaniline can also be used to form core-shell structures.

The surface of these proteins can be further functionalized by surface polymerization, which can further modulate the assembly process and interaction with the biomacromolecules.

### IV. Delivery of Core Materials

As stated, other biologically active materials may be incorporated into the core of the core-shell nanocomposite. Upon disassociation of the bionanocomposite, such as through an increase in pH as discussed above, the bionanocomposite can not only release the shell biomacromolecules from the polymeric core but also can release any other contents of the core.

The core material can be any biologically active material suitable for encapsulating within the bionanocomposite. Particular examples of such biologically active materials include, but are not limited to, drugs and other pharmaceutical agents, vitamins, etc. and combinations thereof.

Thus, the bionanocomposite can be utilized to define and control the delivery rate of a biologically active material. The ability to provide controlled, sustained delivery of biologically active agents to a biological system is desirable to better regulate the pharmacodynamics and efficacy of therapeutic agents delivered in clinical settings as well as to encourage healthy tissue growth and formation in tissue engineering applications. For instance, sustained release delivery methods and systems such as those disclosed herein that can protect drugs from in vivo degradation and/or provide slow, well-controlled, and localized drug delivery to individual patients have numerous advantages over more traditional delivery methods.

### EXAMPLES

### Example 1

Materials: Poly(4-vinylpyridine) (P4VP) (Mw 60,600, 160.000 Da), Poly(2-vinylpyridine) (P2VP) (Mw 6,500, 12,000, 21,000, 47,000, 64,000 Da) copper bromide (CuBr), hose spleen ferritin, acrylic acid, 4-vinylpyridine, N-hydroxysuccinimidyl, PEG methacrylate were purchased from Sigma-Aldrich company and used as received. Water (18.2 MΩ) was drawn from Milli-Q system (Millipore). Poly(2-vinyl pyridine-b-ε-caprolactone) (P2VP₁₉₈-b-PCL₃₁₀) (the subscripts indicate the block lengths; PDI=1.8), poly(styrene-b-4-vinyl pyridine) (PS_{9.8}-b-P4VP₁₀) (PDI=1.08), poly(styrene-b-2-vinyl pyridine) (PS₁₃-b-P2VP_{42.5}) (PDI=1.07), poly(ethylene oxide-b-4-vinyl pyridine) (PEO₅-b-P4VP₂₀) (PDI=1.30), poly(styrene-b-4-vinylpyridine-b-styrene)(PS_{5.3}-b-P4VP₅₈-b-PS_{5.3}) (PDI=1.8), poly(4-vinyl pyridine-b-styrene-b-4-vinyl pyridine) (P4VP_{4.5}-b-PS₂₇-b-P4VP_{4.5}) (PDI=1.09), poly(styrene-b-4-vinyl pyridine-b-ethylene oxide) (PS₆₀-b-P4VP₃₂-b-PEO_{39.5}) (PDI=1.2), were purchased from Polymer Source. Polyaniline (Mw 6,500 Da), poly(3-(2-methoxyethoxy)methythiophenes) (PMT) was synthesized with Mw 2,100. Hemoglobin (Hem) (from human), Gelatin (Gel B) (from bovine skin, Type B), Concanavalin A (Con A) (from Canavalia ensiformis (Jack bean), Type IV), a-chymotrypsin (ChT) (from bovine pancreas, Type II), Avidin (Avi) (from egg white), Ovabumin (Ova) (albumin from chicken egg white, Grade II), Lipase (Lip) (from candida rugosa), Papain (Pap) (from papaya latex), Ribonuclease A (Rib A) (from bovine pancreas), Trypsin (Try) (from bovine pancreas, Type I), Ferritin (Fer) (from horse spleen, Type 1), Mucin (Psm) (from porcine stomach, Type II), pepsin (Pep), and cytochrome c (Cyt) were purchased from Sigma-Aldrich company. Apo lipoprotein E (Apo E4) (from Human AG) was purchased from Fitzgerald Industries International. Bovine serum albumin fraction V (BSA) and Lysozyme (Lys) (from Hen Egg White) were purchased from Rockland company. Horseradish Peroxidase (HRP) and Strepavidin (Str) were purchased from Thermo scientific. Albumin from human serum (HSA) was purchased from Fluka.

Purification: Tobacco plants approximately 1 month old were inoculated with wt-TMV. The leaves were harvested, and the virus was isolated from the host plant. Briefly, the leaves were crushed and blended with 0.01 M K-phosphate buffer at pH 7.8 with 0.2% p-mercaptoethanol. The mixture was centrifuged at 9000 rpm for 15 minutes after which the supernatant was clarified with 1:1 CHCl3:1-butanol. The aqueous portion was separated by centrifugation, and TMV was precipitated by the addition of PEG 8K to 10% and NaCl to 0.2 M. The resulting pellet was resuspended in 0.01 M K-phosphate buffer at pH 7.8. After a final round of ultracentrifugation at 42 k rpm for 2.5 hours, the resulting pellet was resuspended overnight in 0.01 M K-phosphate buffer at pH 7.8 or in pure water.

The procedure for purification of TYMV and CPMV was similar with that TMV.

Assembly of Polymer with Proteins or BNPs:

a. Synthesis of CPMV-co-Polymer

A solution of P4VP (Mw 60,000 Da), P2VP (Mw 150,000 Da) and PS-b-P4VP (in DMF (2.0 mg/mL, 0.5 mL) was slowly added into a solution of CPMV in pure water (6.67 μg/mL, 16.5 mL) under stirring. Then the samples were dialyzed against nanopure water (2×1 L) with the MW 3500 cut-off dialysis tube (from Pierce).

b. Synthesis of TYMV-co-P4VP

A solution of P4VP (Mw 60,000 Da) in DMF (2.0 mg/mL, 0.5 mL) was slowly added into a solution of TYMV in pure water (6.67 μg/mL, 16.5 mL) under stirring. Then the samples were dialysis against nanopure water (2×1 L) with the MW 3500 cut-off dialysis tube (from Pierce).

c. Synthesis of TMV-co-Polymer, M13-co-P4VP

A solution of P4VP (Mw 60,000 Da) in DMF (2.0 mg/mL, 0.5 mL) was slowly added into a solution of TMV or M13 in pure water (6.67 μg/mL, 16.5 mL) under stirring. Then the samples were dialyzed against nanopure water (2×1 L) with the MW 3500 cut-off dialysis tube (from Pierce). A solution of P4VP-b-PS-b-P4VP in DMF or dioxane (2.0 mg/mL, 0.5 mL) was slowly added into a solution of TMV in pure water (6.67 μg/mL, 16.5 mL) under stirring. Then the samples were dialyzed against nanopure water (2×1 L) with the MW 3500 cut-off dialysis tube (from Pierce).

d. Synthesis of Protein-co-P4VP, Protein-co-P2VP

A solution of P4VP or P2VP (Mw 60,000 Da) in DMF (2.0 mg/mL, 0.5 mL) was slowly added into a solution of different proteins in pure water (6.67 μg/mL, 16.5 mL) under stirring. Then the samples were dialysis against nanopure water (2×1 L) with the MW 3500 cut-off dialysis tube (from Pierce).

4. Surface Polymerization of CPMV-P4VP, 4VP-CPMV (More in Detail)

a. In a typical experiment, purified 4-vinlypyridine (1 μl/ml) was introduced to an aqueous CPMV solution (0.5 mg/ml), followed by addition of ammonium persulfate (0.01 mg/ml). The total volume of the solution is 1 ml. The PH of the reaction was around 6.5. Then the solution was purged with nitrogen and the reaction was incubated at room temperature for overnight. After the reaction, the solution was purified by dialysis upon nanopure water with MW 100,000 dialysis tube. The experiment details are summarized as following. 4-vinlypyridine (20 μL/mL) was introduced to an aqueous CPMV solution (2 mg/ml), followed by addition of ammonium persulfate (2 mg/ml). The total volume of the solution is 1 mL.

b. Synthesis of TMV-Poly(HEMA) TMV is purified according to the procedure reported. The polymerization procedure is similar to the apoferritin-PEGMA [4d]. The initiator was obtained by click chemistry. In a typical polymerization reaction, TMV macro-initiator (1 mg, 5.5×10-5 mmol) and HEMA (8.2 mg, 0.066 mmol) were dissolved in degassed water (200 μL) with N2 purge for 30-60 min. A 400 μL of a catalyst stock solution (obtained by mixing 14.3 mg CuBr, 27.5 mg 2,2/-bipyridine in 4 mL degassed water) was added to initiate the polymerization. The reaction proceeded under nitrogen for 90 min before the mixture was exposed to air and diluted with 200 μL of water to quench the reaction. The product was purified by dialysis against nanopure water with 10,000 MW cut-off membranes.

5. Analysis. TEM analysis was carried out by depositing 20 μL aliquots of each sample at a concentration of 0.1 to 0.5 mg mL-1 onto 100-mesh carbon coated copper grids for 2 minutes. The grids were then stained with 20 μL of uranyl acetate and observed with a Hitachi H-8000 electron microscope. For FESEM analysis, the sample was dried overnight and coated with Pt, then check by a Hitachi S4800 electron microscope. The DLS analysis was performed using Submicron Particle Sizer AutodillutePAT Model 370. SAXS of solutions were measured at Sector 12, Advanced Photon Source in Argonne National Laboratory. X-ray beam of energy 12 keV and flow cell equipped with 2 mm thick quartz capillary were used.

### IV. Some Typical Results

A. Hierarchical Co-Assembly of CPMV and Polymer

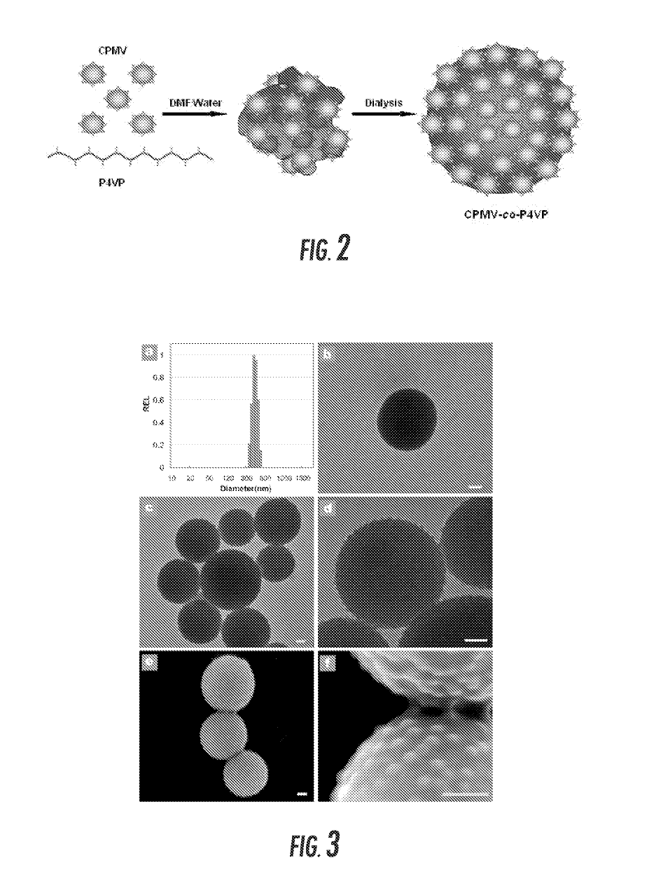

As shown in FIG. 2, the CPMV-co-P4VP nanocomposite was obtained by mixing CPMV aqueous solutions and P4VP to prepare the biocomposite spheres with CPMV.

In a typical experiment, a 2.0 mg/mL solution of P4VP (Mw 60,000 Da) in dimethylformamide (DMF) was slowly added to a solution of CPMV in pure water with stirring. The final concentration of CPMV was 0.006 mg/mL. For the sample CPMV-co-P4VP, with the mass ratio M_{CPMV}/M=of 0.055, a thorough dialysis against water made the solution turn faintly blue and opalescent while no precipitation of P4VP was observed, indicating the formation of colloidal assemblies. Without CPMV, most of the P4VP precipitated and the solution was transparent and colorless. Transmission electron microscopy (TEM), field emission scanning electron microscopy (FESEM) and dynamic lighter scattering (DLS) were used to characterize the structure of CPMV-co-P4VP. As shown in FIG. 3a, the hydrodynamic diameter of the biocomposite spheres is in the range of 723±340 nm as measured by DLS. Upon negative staining using uranyl acetate, TEM shows the presence of CPMV particles on the surface of P4VP spheres (FIG. 3c, 3d), which is particularly clear in the edge area. As comparison, P4VP alone has much fewer particles remaining in solution and all particles have smooth surfaces (FIG. 3b). The coverage of viral particles can be better observed using FESEM (FIG. 3e, 2f). The average size of CPMV is about 33 nm, a little bit larger than the solution diameter of CPMV (˜29 nm), which may be caused by the gold splashing. Additionally, the drying and coating process for SEM measurement may also deform the virion. The size of most raspberry-like structures observed from TEM and FESEM images is in the range of 400-700 nm, which is smaller than that obtained from DLS results. Likely CPMV-co-P4VP may shrink at the dry state.

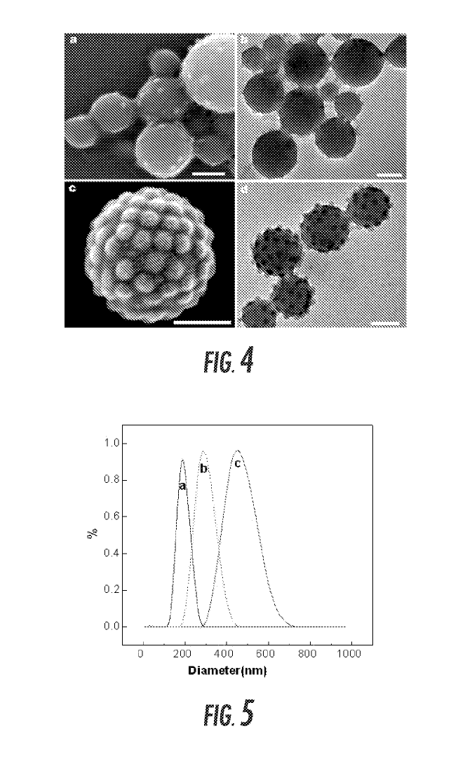

Since P4VP has both hydrophilic and hydrophilic motifs, the solvent composition always plays an important role in its morphological transformation. Some recent studies showed that different solvents could totally reshape the P4VP-copolymer assemblies. It was found that dialysis against water may greatly change the morphology of CPMV-co-P4VP aggregates. Shown in FIG. 4a-b are the FESEM and TEM images of CPMV-co-P4VP composites with mass ratio M_{CPMV}/M_{P4VP} of 0.11 before dialysis. Although the aggregates show raspberry-like features, the coverage of CPMV on the P4VP spheres is very low. With dialysis, the viral coverage dramatically increased (FIG. 4c, d). In addition, many isolated CPMV particles were observed in solution before dialysis. The morphological change before and after dialysis can be explained by that the energy associated with the non-favorable interactions between the DMF/water co-solvent and P4VP is smaller than between pure water and P4VP. So after dialysis, more viral particles are needed to cover the P4VP spheres in order to decrease the interfacial energy.

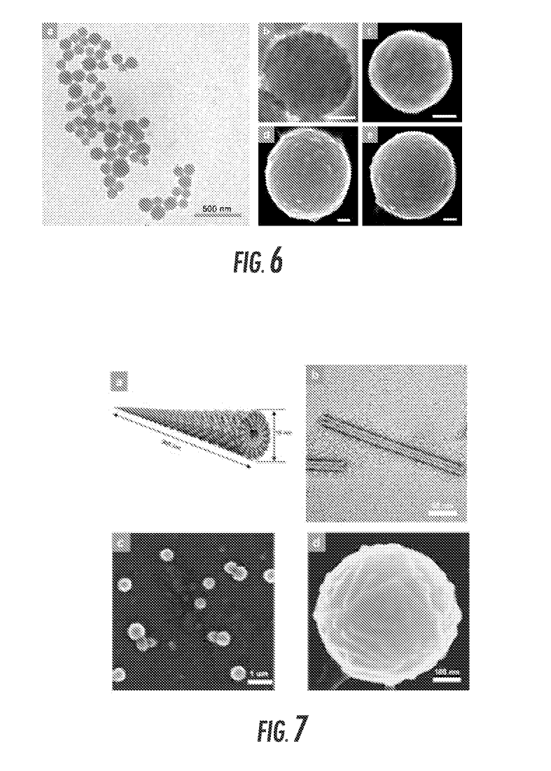

As a test, different mass ratio of CPMV and P4VP (M_{CPMV}/M_{P4VP}) was used to control the size of final CPMV-co-P4VP assemblies. While the amount of CPMV is fixed, the more P4VP is used in the reaction, the larger are the composite particles. Three samples with different mass ratio were prepared and analyzed using DLS. As shown in FIG. 5, the mean hydrodynamic diameter is 723, 298, and 199 nm for MCPMV/MP4VP=0.055, 0.11 and 0.367, respectively. These results are consistent with the TEM and FESEM analysis that while more P4VP added in the reaction, the size of the final aggregates increased accordingly to achieve the full viral coverage. The theoretical model of the raspberry-like packing is related to the size of viral particle, size and curvature of the sphere, and the repulsion between viral particles was established which was consistent with experimental data.

Using the same protocol, other biological spherical nanoparticles were used to fabricate similar raspberry structures. Horse spleen ferritin (HSF) is a 12-nm iron storage protein cages composed of a self-assembled protein shell and a ferrihydrite core. FIG. 6a shows the TEM image of the composite spheres synthesized by co-assembly of HSF and P4VP with mass ratio of M_{Ferritin}/M_{P4VP} of 0.3. Despite the morphology of HSF, it can not be observed easily due to its smaller size; however, the raspberry-like structure of the assembly can be clearly distinguished (shown in FIG. 6b). The dark dots displayed on the surface of the composite spheres are attributed to the high electron density of the iron oxyhydroxide cores of HSF. FESEM images (FIG. 6c-e) also show the ferritin particles coated on the P4VP spheres. The diameter of the ferritin particles is about 14 nm.

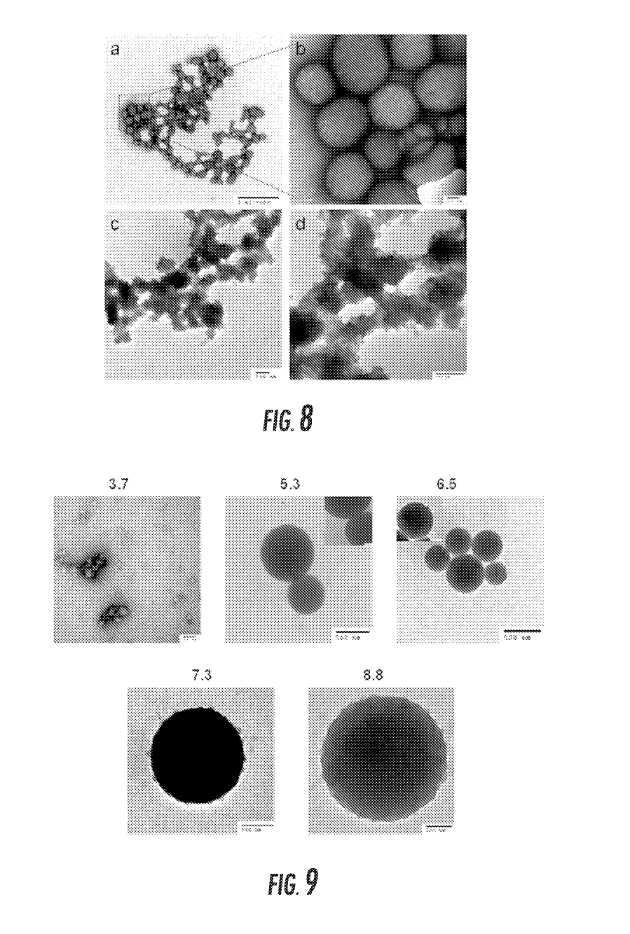

Moreover, it was found that the rod-like bioparticles such as tobacco mosaic virus (TMV) can also be used to fabricate the core-shell biocomposites. As shown in FIG. 7b, the diameter of composite spheres is in the range of 400-800 nm which is similar with that CPMV-co-P4VP with mass ratio M_{TMV}/M_{P4VP} of 0.2. TMV particles can be clearly observed by a magnified image (FIG. 7c-d).

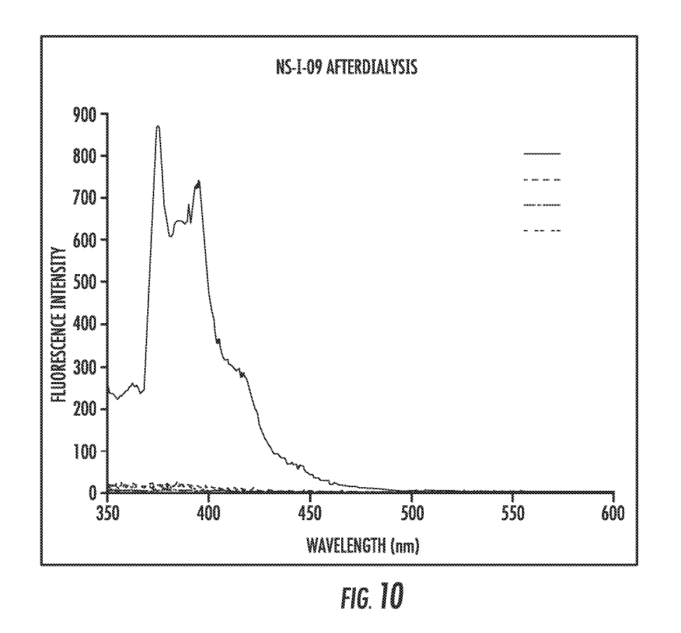

This approach is applicable to other types of polymers and biomacromolecules. FIG. 8a shows the TEM images of CPMV-co-P2VP. FIG. 8b is the enlarged images, which clearly demonstrate that CPMV is covered on the 4VP sphere. This is explained by the negative agent selectively staining the virus to make it clear. In addition, the diblock copolymer PS-b-P2VP can also be used as the building block. FIG. 8c-d shows the TEM images of CPMV-co-PS-b-P2VP biocomposites. Clearly, the CPMV is coated on the surface of PS-b-P2VP.

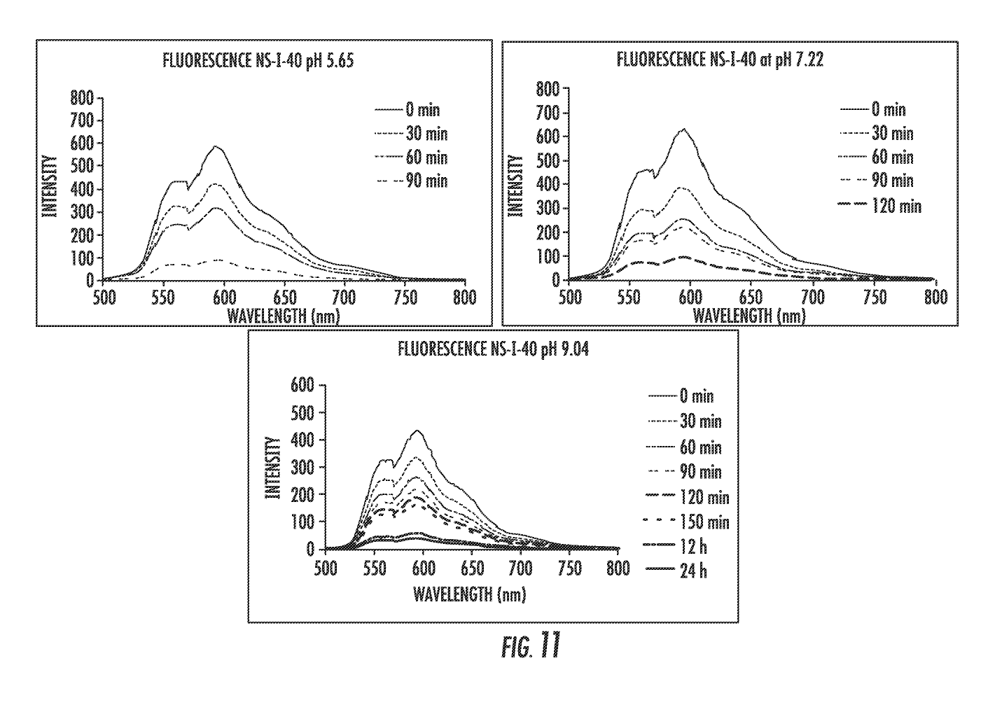

The dye can be encapsulated into the CPMV-co-P4VP composites. The results are shown in FIG. 10. A is the system only with DMF and water. B is the system with CPMV in DMF/Water system. C is the system with P4VP in DMF/Water system. D is the system with CPMV-P4VP in DMF/Water system. After dialysis, only the D system shows the fluorescence of pyrene, which can confirm that the pyrene is encapsulate into the CPMV-P4VP.

Meanwhile, DOX was encapsulated into those CPMV-co-P4VP nanoparticles, and the drug release was evaluated, as shown in FIG. 11. Triethylamine (TEA) was conjugated with DOX to increase the hydrophobicity of DOX. The formation of DOX-DOX increases the hydrophobicity of the drug itself and also increases the drug uptake into a polymeric micelle. The yield of DOX loading into CPMV-P4VP was around 90% based on the initial amount of DOX. The release of DOX from CPMV-co-P4VP was examined at three different conditions: neutral and acidic conditions since the tumor cells are slightly acidic. At neutral condition, DOX was released relatively slow, and the rate was almost stable afterwards. In contrast, under acidic condition, DOX was released rapidly in the first hour and constantly decreased thereafter. It is believed that the release of DOX from nanoparticles might be controlled by the diffusion. The result clearly indicated that the release of DOX was retarded significantly under a physiological condition, which suggested that the micelles could be used as a controlled release carrier for the poorly soluble drug.

B. Hierarchical Co-Assembly of TYMV and Polymer

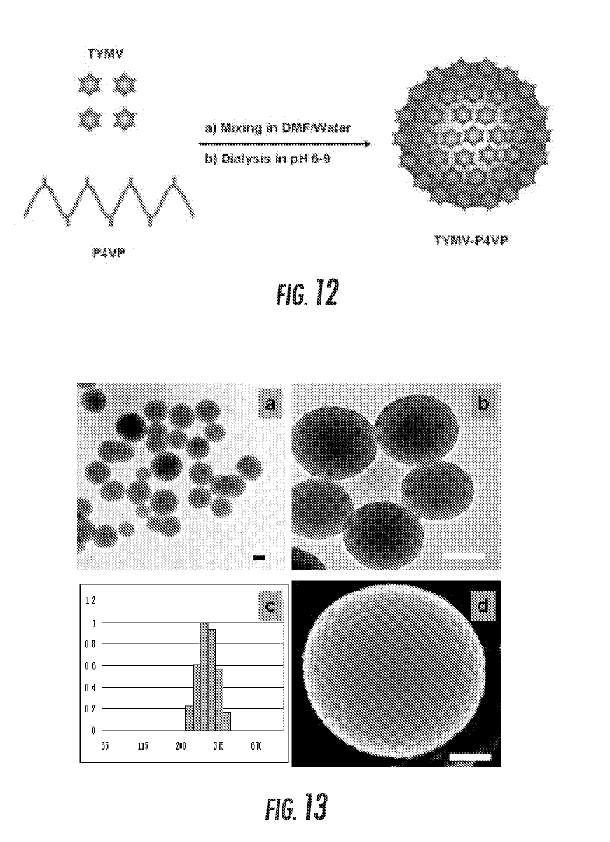

FIG. 12 shows a general pathway to generate the hybridized composite colloids (TYMV-co-P4VP) by mixing TYMV with P4VP at neutral pH. In a typical experiment, 500 μL of 2.0 mg/mL solution of P4VP (Mw 60,000 Da) in dimethylformamide (DMF) was slowly added to a solution of TYMV in pure water with vigorous stirring. The final concentration of P4VP was 0.006 mg/mL. The final product was dialyzed against pure water.

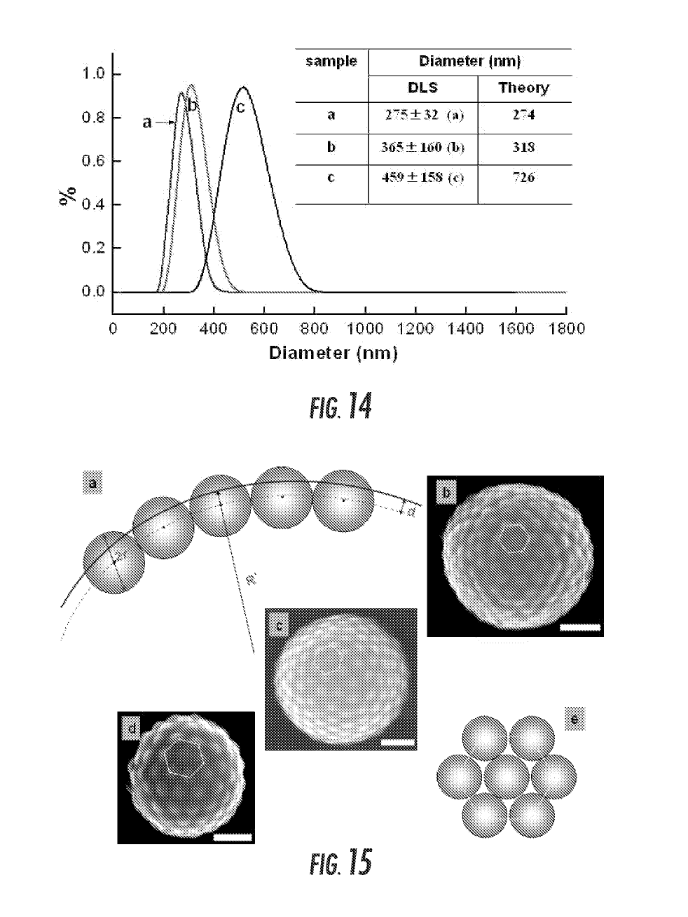

Transmission electron microscopy (TEM), field emission scanning electron microscopy (FESEM) and dynamic light scattering (DLS) were used to characterize the structure of the final product. Upon negative staining with uranyl acetate, spherical TYMV-co-P4VP colloids can be detected using TEM (FIG. 13a). It was also observed that TYMV particles are coated on the exterior surface of the colloids which is clearly seen by the roughing of the spherical edges (FIG. 13b). As a comparison, P4VP alone shows many fewer particles in solution and all of the polymeric particles have very smooth surfaces. The hydrodynamic diameter of the assemblies range around 365±160 nm based on DLS measurements (FIG. 13c). The coverage of viral particles can be better observed under FESEM, and the surface of the composite colloid is fully covered with TYMV particles to give a raspberry morphology (FIG. 13d). Moreover, TYMV particles form an ordered pattern with hexagonal stacking. The average distance between centers of two viral particles is about 31 nm, consistent with the diameter of TYMV (˜28 nm) which maybe caused by Pt splashing.

The size of the final TYMV-co-P4VP assemblies can be controlled by varying the mass ratio of TYMV and P4VP (M_{TYMV}/M_{P4VP}) As shown in FIG. 14, the mean hydrodynamic diameter is 275, 365, and 548 nm for MTYMV/MP4VP=0.3, 0.2 and 0.1, respectively. These results are consistent with the TEM analysis. It was observed that when more P4VP is added in the reaction, the size of the final aggregates increased accordingly to achieve the full viral coverage.

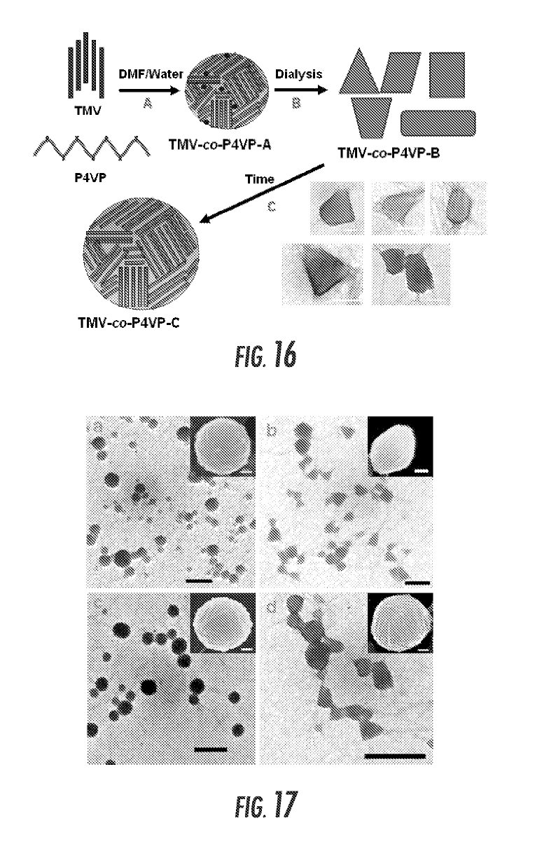

Based on the FESEM images (FIG. 15a-c), it can be clearly seen that different sized TYMV-co-P4VP assembles are all covered with TYMV particles that are close-packed with a hexagonal pattern (FIG. 15d). In addition, it can be roughly estimated that about two thirds of the TYMV sphere is embedded in the P4VP sphere. Therefore, a simplified mathematical model can be used to simulate experimental results. First the following considerations were made: a) all of the TYMV-co-P4VP assembles are always fully covered with TYMV; b) there are no free TYMV particles remaining in the solution; c) both TYMV and TYMV-co-P4VP are monodisperse; d) TYMV has 2-D square lateral packing on the surface; and e) the density of P4VP is equal to density of 4-vinylpyridine monomer. FIG. 15e depicts the mathematical model, where d is the depth of TYMV particles embedded in the P4VP, r is the radii TYMV, and R′ is the appeared radii of the final assembly. Details of the calculation are included in the supporting information. Based on the model established, the theoretical diameters of TYMV-co-P4VP particles are 274, 412 and 770 nm respectively when M_{TYMV}/M_{P4VP} ratios are 0.3, 0.2 and 0.1, which are in nice agreement with the experimental data (inset Table of FIG. 15).

C. Hierarchical Co-Assembly of TMV and Polymer

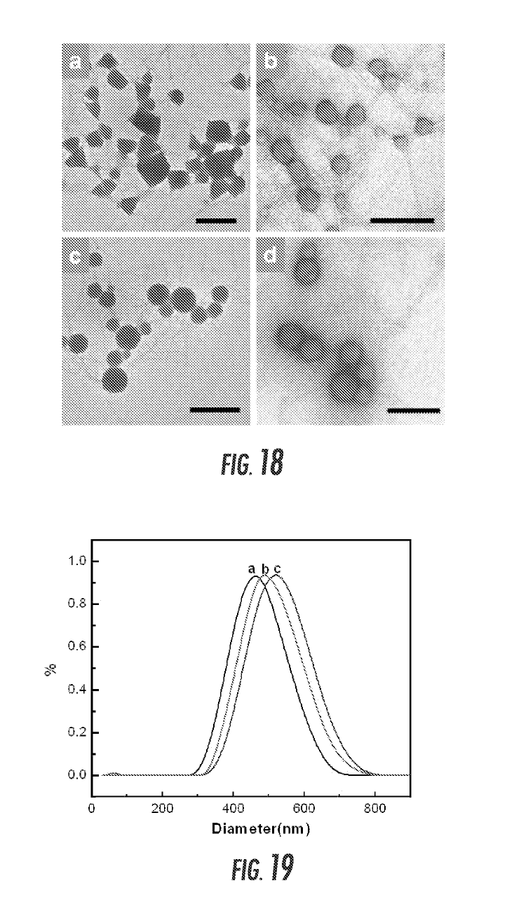

As shown in FIG. 16, TMV-co-P4VP nanocomposites with different shapes can be easily obtained by simple mixing TMV aqueous solution and P4VP. Based on FIG. 16 there are three steps to farm the final TMV-co-P4VP composites. Firstly, pathway A is used to form spherical TMV-co-P4VP-A by mixing of TMV and P4VP in DMF/water solution. After dialysis, the colloids of TMV-co-P4VP-A change their shape to TMV-co-P4VP-B with irregular shape such as triangle, trapezoid, rectangular, and parallelogrammical. Surprisingly, after longer time, TMV-co-P4VP-B change to spherical again, called TMV-co-P4VP-C.

Transmission electron microscopy (TEM) and field emission scanning electron microscopy (FESEM) were used to characterize the structure of TMV-co-P4VP. Upon negative staining using uranyl acetate, TEM (FIG. 17a-d) shows different morphologies of TMV-co-P4VP-A-C at different stages. FIG. 17a shows the TEM images of TMV-co-P4VP-A with spherical morphology. Inset picture shows the FESEM images of TMV-co-P4VP-A. It seems that they are formed by either attaching or inserted to the P4VP spheres. Surprisingly, the morphology of TMV-co-P4VP-B is irregular shape with triangle, trapezoid, rectangular, and parallelogrammical, as shown in FIG. 17b. Inset is the FESEM image of TMV-co-P4VP-B, which clearly shows the surface morphology. After dialysis, and keeping for longer time, it can be observed that the morphology changes to spherical-like again, shown in FIG. 17c, Inset image is the individual particle. In the process of dialysis, in checking the intermediate sample, the sample morphology changes from spherical to irregular shapes after short time, as shown in FIG. 17d. The mechanism of this morphology transition could be explained from different equilibrium state. Before dialysis, TMV-co-P4VP-A is equilibrium consisting of four phases of P4VP, TMV, DMF, water, in which P4VP is likely to become the spherical to reach the equilibrium while the TMV attached or inserted into the P4VP sphere (Based on the FESEM images). However, after dialysis, the equilibrium of four-phase system is destroyed. As the content of DMF decreases, TMV is likely to keep its rod-like shape, induce the self-assembly behavior of P4VP to make irregular shapes appear. In another words, rod-like TMV plays an important role of re-assembly of P4VP in this state. It should be noted that the stage of forming irregular shapes can be considered to be semi-equilibrium state, which finally should be transfer to thermo-equilibrium state. Therefore, after longer time, the TMV-co-P4VP-B is transferred to TMV-co-P4VP-C with spherical shape.

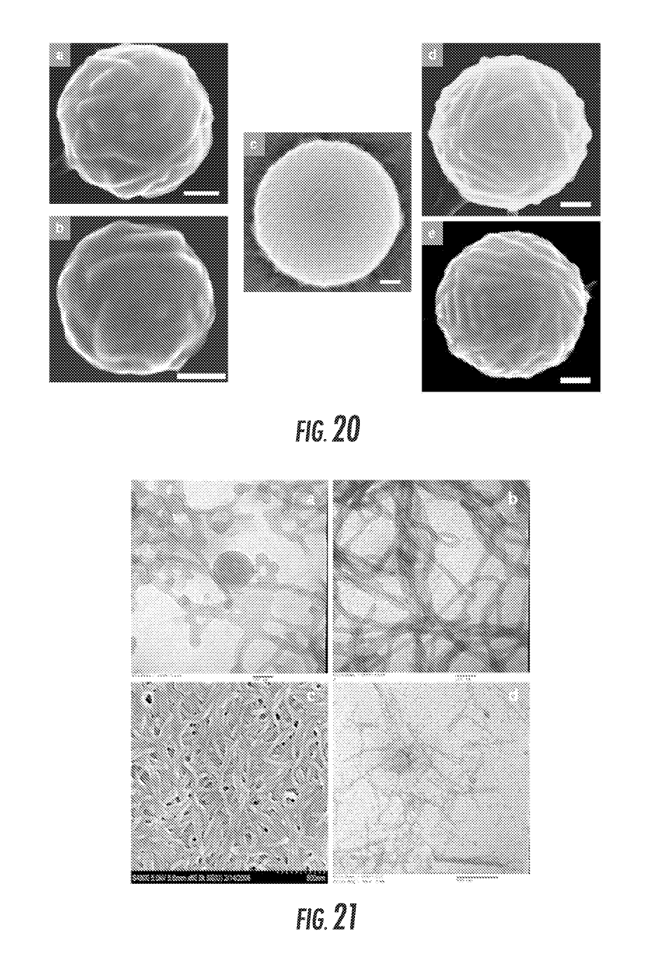

It is noted that solvent and time have effects on the morphology transition. In order to confirm this, aggregation of both TMV-co-P4VP-A and TMV-co-P4VP on the path B to the final biocomposites was observed by taking aliquots from the reaction mixtures at various times and recorded using TEM (FIG. 18). At very early stages of TMV-co-P4VP-A with 3% DMF, the samples consist of spherical particles. By checking different times, it was found that the shapes keep spherical and stable (FIG. 18d). However, for the TMV-co-P4VP-B, different results were observed. At the early stages 0 day, the shape is irregular (FIG. 18a). The irregular shape particles begin to aggregate into practically spherical in the next stage after 3 days (FIG. 18b). As time increases to 7 d, the number of aggregates with spherical shapes increases (FIG. 18c). The only difference of TMV-co-P4VP A and C is the content of DMF. It appears that the morphology changes are controlled by the DMF and time. Therefore, the DMF content was lowered and the morphology of samples was checked. At the 3%, 0.3%, 0.03% DMF content, it was found that the morphology is still mainly spherical with the increase of time. Therefore, the unique sphere-to-triangle-sphere transformation is very complicated including destroyed equilibrium and decrease of DMF content and longer time to reach the final equilibrium.

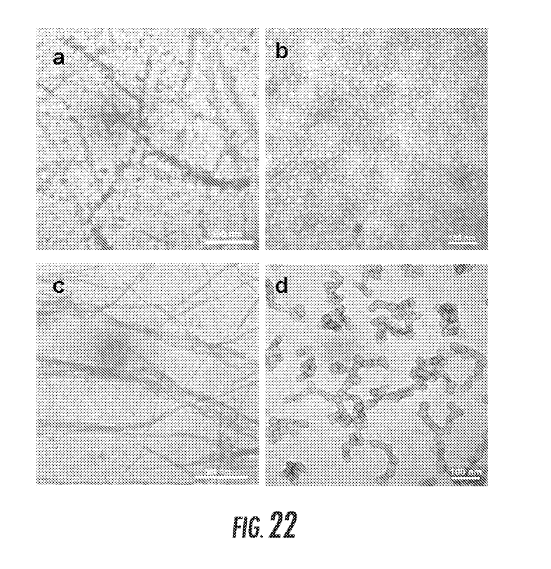

Dynamic light scattering (DLS) (shown in FIG. 19) is a useful technique to characterize the size of colloidal particles. Similar to the other raspberry-like virus-co-polymer, the size of the final TMV-co-P4VP-C assemblies can be controlled by the mass ratio of TMV and P4VP (M_{TMV}/M_{P4VP}). While the quantity of P4VP is fixed, the more TMV that is used in the reaction, the smaller are the composite particles. Three samples with different mass ratio were prepared and analyzed using DLS. The mean hydrodynamic diameter is 655, 564, and 456 nm for M_{TMV}/M==0.20, 0.30 and 0.50, respectively. These results are consistent with the TEM analysis

It has been demonstrated that surface curvature of cores play an important role on the self-assembly of virus. In addition, M13 virus can form nano-ring like morphology by using two genetic modifications encoding binding peptides in the presence of linker. Rod-like gold nano-wire can assemble on the surface of polymer surface. However, it was found that gold particle cannot bind to the surface of polymer. Here, similar and different results appear. First, it was found that single TMV virus can bend without denature in the high curvature of polymer sphere. For smaller colloids of the particle size ranging from 200-400 nm (FIG. 20a-b), many TMV viruses bend to the polymer, which arise from an increase of strain energy and some decrease in entropy. Non-covalent interactions may contribute to overcome the required activation energy to bend the virus. As the size of colloids become progressively larger than 500 nm (FIG. 20c-e), the TMV is covered by TMV with intact shape, even with little bending of TMV.

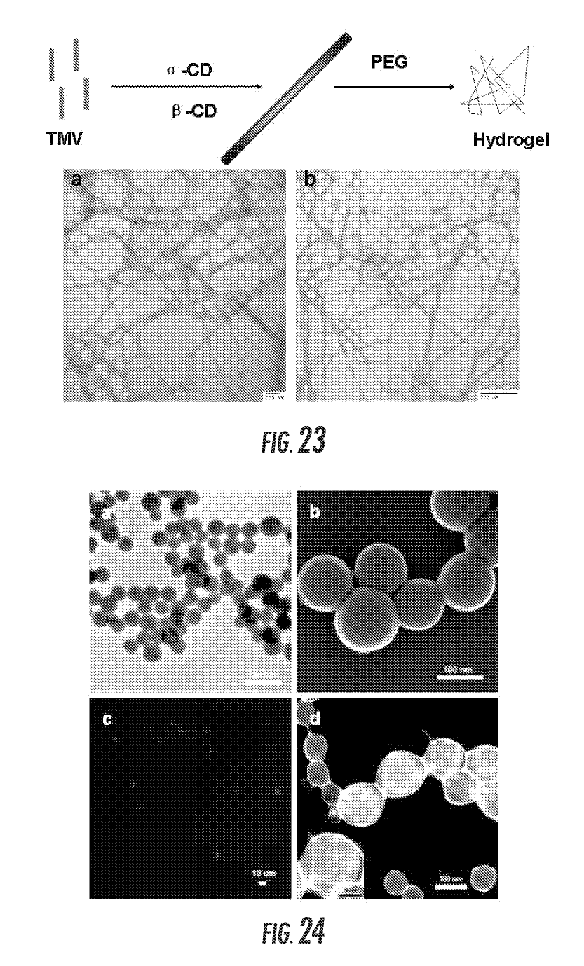

Another interesting factor is that TMV can be used to control the phase behavior of triblock P4VP-PS-P4VP behavior. The morphology of P4VP-PS-P4VP micelles is multiple including micelles, vesicles, and branched rod (shown in FIG. 21a). However, after the addition of TMV, it can be found that only the TMV rod-like morphology without branched and spheres, which indicates that the TMV induce the phase change (FIG. 21b). This behavior can be confirmed by the FESEM images (FIG. 21c). In addition, by controlling the order of addition of TMV, it can found that some different morphology formed. More small micelles with size ranging from 30-50 nm can be coated on the TMV tubes.

It was found that the morphology of the sample will have a big change when the triblock copolymer was added to the water or TMV solution. As shown in FIG. 22 a,c, when triblock copolymer in dioxane and DMF was added to the TMV solution, the morphology of the formed composited is TMV coated with ball and long fiber, respectively. However, for the control experiment, in FIG. 22 b,d when triblock copolymer in dioxane and DMF was added to water solution, the morphology of the particles is spherical with different sizes. It indicates that the solvent has a big effect on the assembly of the TMV and triblock copolymers. The formed long fiber can be considered as a good template to facilitate the growth of inorganic nanoparticles to form the metallic nanowires.

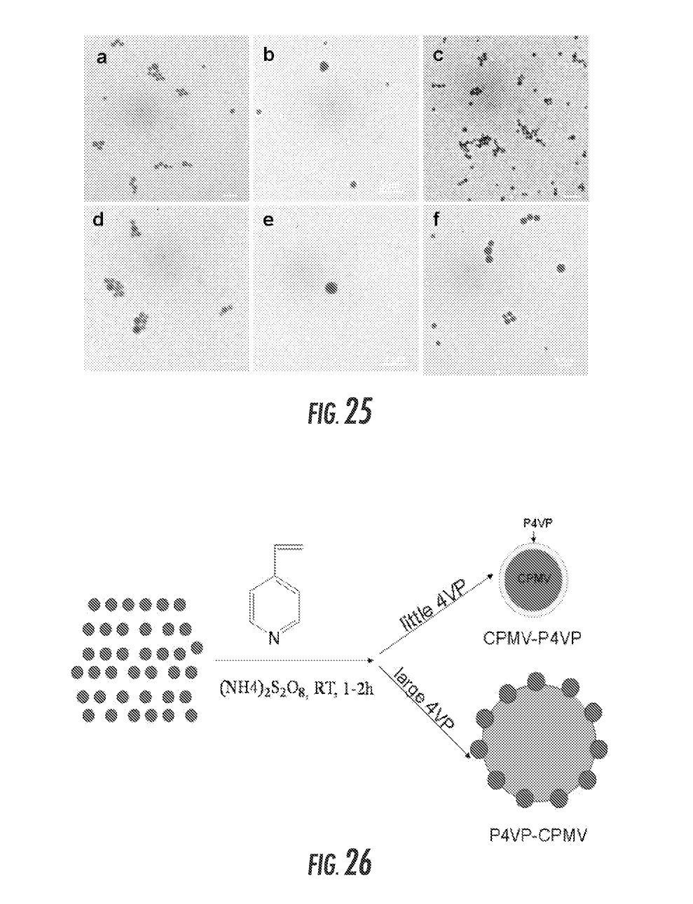

Using the similar method, TMV can form the long fiber with CD due to their non-covalent interaction. TEM images are shown in FIGS. 23a-b. The most interesting thing is that they can form the three dimensional structures hydrogel by adding the PEG into the formed long fiber. The hydrogel is a good scaffold for the drug delivery and cell culture study.

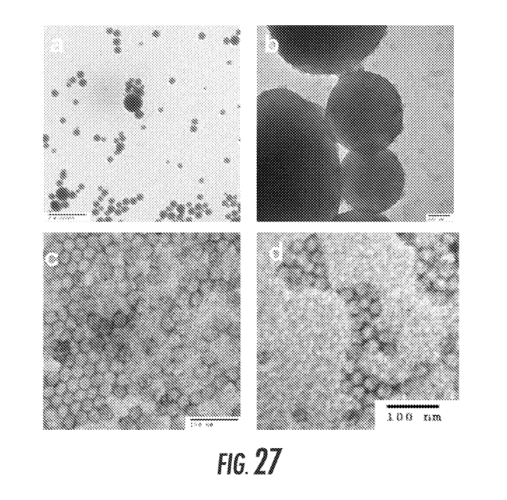

A similar method can be applied to other rod-like biological particles such as M13 bacteriophage, which is around 6.5 nm in diameter and 880 nm in length. M13 is composed of 2700 major coat proteins helically stacked around its single-stranded DNA. When M13 is co-assembled with P4VP in solution, it was observed that M13-co-P4VP formed with spherical shape, which can be attributed to the flexibility of M13 compared to TMV. However, different from TMV-co-P4VP, the edges of M13-co-P4VP are very smooth. TEM and SEM images (FIG. 24a-b) can not show the single M13, given that 6.5 nm is the limit of FESEM for biological sample. In order to confirm that M13 is coated on the surface of P4VP, M13 was modified with rhodamine dye. UV-vis, MALDI-TOF MS, and fluorescent microscopy showed that M13 was modified by rhodamine dye (data not shown). The fluorescence microscopy images showed the appearance of a little red spherical droplet, as shown in FIG. 24c, which can be attributed to the modified M13. In order to confirm that M13 is still intact, dark field TEM is used to observe the morphology. It can be clearly observed that many M13 viruses covered the P4VP sphere (FIG. 24d).

D. P4VP Co-Assembly with Different Proteins.

Besides the above virus particles, another 10 different proteins can be chosen as the building blocks, shown in Table 1. These proteins possess diverse structural characteristics such as metal/nonmetal-containing, molecular weight (Mw), isoelectric point (PI), and UV absorbencies. Notably, these proteins have comparable Mw and PI values, thereby providing excellent objects for examining the ability of P4VP. Using P4VP as the building block, it can be used to stabilize the listed proteins. FIG. 25 shows the TEM images of different protein-co-P4VP micelles. Notably, the PI has some effects on the colloid assembly, where low PI between 4 and 6 can facilitate the assembly. Further experiments are being conducted to demonstrate the robustness of the method. In particular, the protein functionality is being tested after the formation of the protein-co-P4VP colloids.

TABLE 1
Basic Properties of the Proteins Used as Building Blocks

| Protein | Mw (kDa) | pI | P4VP |
|---|---|---|---|
| Bovine serum albumin (BSA) | 66.3 | 4.8 | C |
| α-chymotrypsin (ChT) | 25.0 | 8.5 | C |
| Ferritin (Fer) | 750.0 | 4.5 | C |
| Hemoglobin (Hem) | 64.5 | 6.8 | C |
| Human serum albumin (HSA) | 69.4 | 5.2 | C |
| Lysozyme (Lys) | 14.4 | 11.0 | A |
| Gelatin type A (Gel A) | 87.5 | 9.2 | A |
| Avidin (Avi) from egg white | 69.0 | 10.5 | A |
| Strepavdin (Str) | 53.0 | 5.0 | C |
| Ribonuclease A (Rib A) | 13.7 | 9.4 | A |
| Trpsin (Trp) | 23.3 | 10.5 | A |
| Ovabumin (Ova) | 45.0 | 5.1 | C |
| Concanavlin A (Con A) | 104.0 | 4.5 | C |
| Porcine stomach mucin (Psm) | 103.0 | 4.4 | C |
| Lipase (Lip) | 58.0 | 5.6 | C |
| Gelatin type B (Gel B) | 60.0 | 4.8 | C |
| α-chymotrypsinogenA (ChT A) | 25.7 | 9.0 | A |
| Papain (Pap) | 23.0 | 9.6 | A |
| Horseradish Peroxidase (HRP) | 40.0 | 7.2 | C |
| Apolipoprotein E4 (Apo E4) | 59.0 | 5.5 | C |

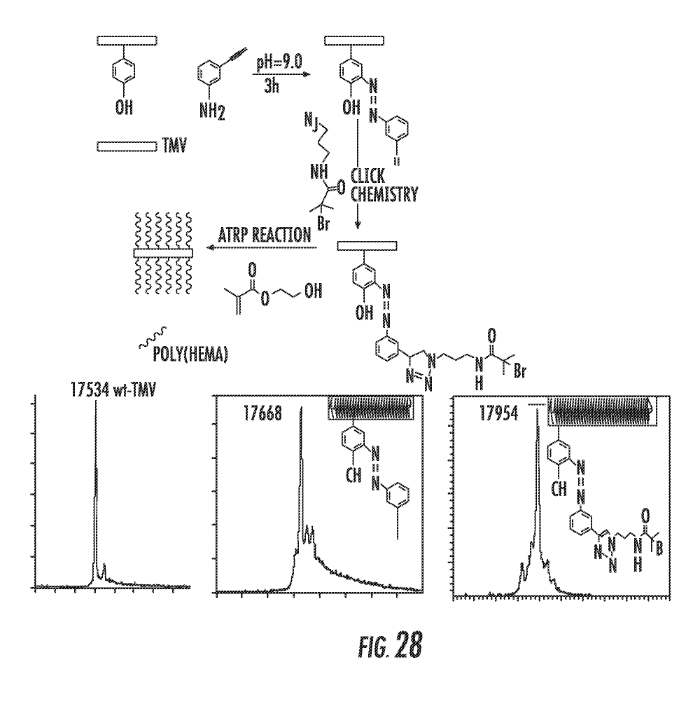

As shown in FIG. 26, one efficient way to form the virus/polymer composites using surface polymerization of virus particles. By adjusting the amount of 4-VP monomer, two different types of structures are observed. When little 4VP added, CPMV-P4VP can be fabricated, with P4VP layer coated at the CPMV surface, However, when a larger amount of 4-VP is added, raspberry-like morphology is obtained. The advantages of this method can be shown as follows: (1) very efficient way to form different morphology of CPMV/Polymer composites, and (2) various monomers can be added to fabricated various diblock copolymer coated virus.

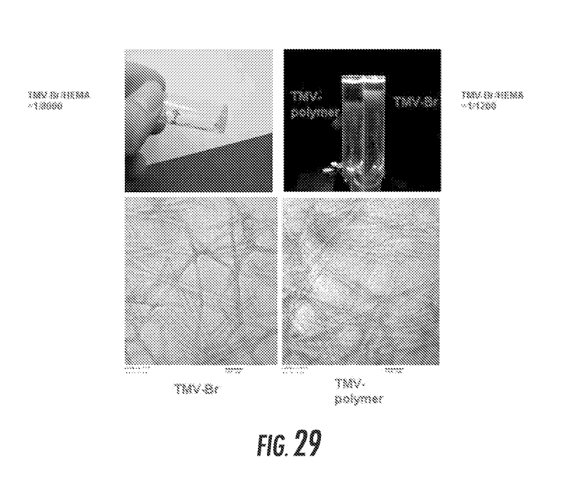

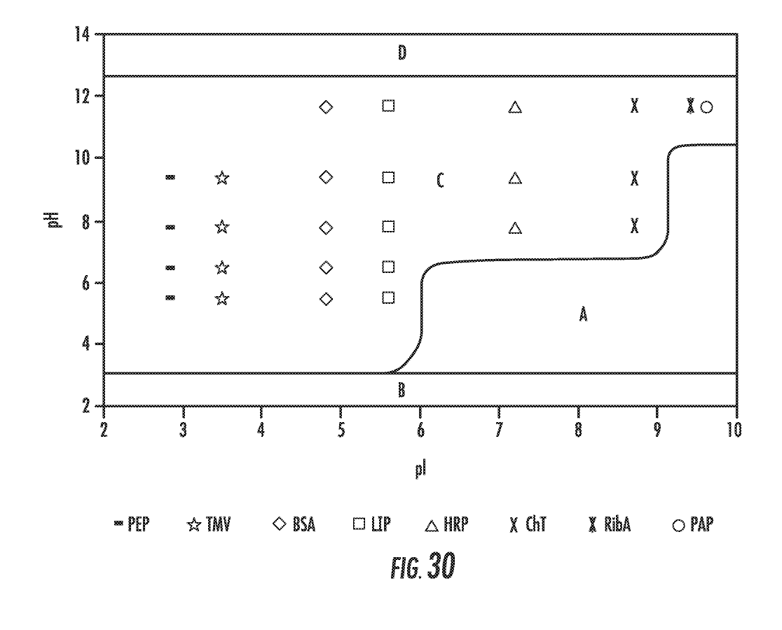

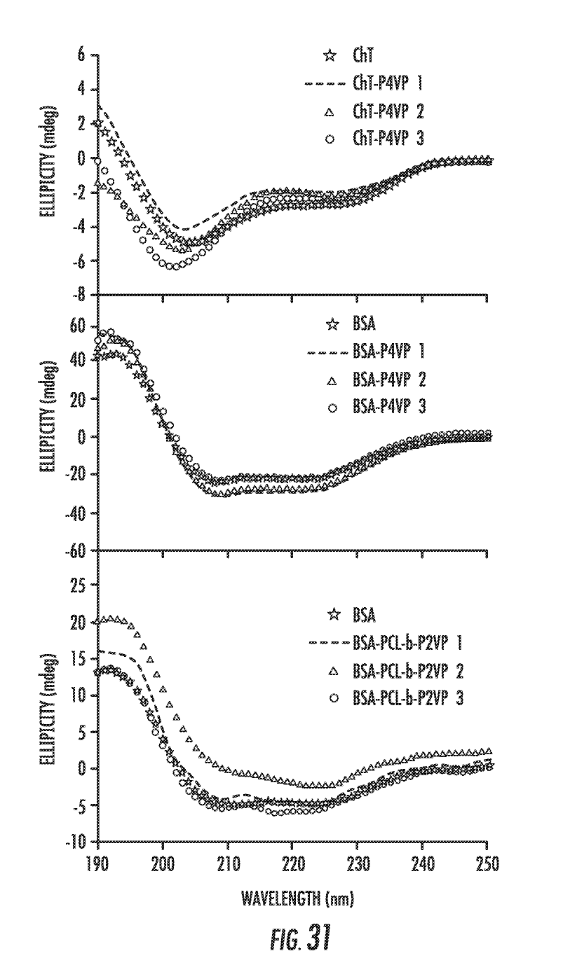

FIG. 27a-d shows typical TEM images of P4VP, CPMV-P4VP, P4VP-CPMV biocomposites by surface modification of polymer. In case of 4VP polymerization, it can be found that they form the spherical particles, with very smooth surface, shown in FIG. 27a. However, with CPMV added, raspberry-like CPMV-P4VP is formed, similar to the structure observed, shown in FIG. 27b. However, after little 4VP is added, it was found that the size of CPMV increase from 30 nm to 33, 34 nm, respectively

As shown in FIG. 28, MALDI-TOF MS results confirm that the macro-initiator is grafted to the TMV surface. The peak of the native TMV is 17534 m/z. As the TMV is modified with alkyne, the peak increased to 17668 m/z. When the TMV macro-initiator is formed by click reaction, the peak reaches to 17954 m/z, confirming that the bromide is successfully conjugated.

Water soluble 2-hydroxyethyl methacrylate (HEMA) was employed as the monomer, and CuBr/bipyridine complex was used as a catalyst to promote the ATRP reaction. A gel was formed immediately when the molar ratio of initiator and monomer was 1:8000, which could not be re-dissolved with a large quantity of water, dichloromethane (DCM), even DMF (FIG. 29a). This can be explained by crosslinking of the polymer. In order to obtain un-crosslinked TMV-Poly(HEMA), the ratio of initiator and monomer is changed to 1:1200. The resulting biocomposite was checked using sucrose gradient sedimentation. As shown in FIG. 29b, it can be observed that the layer of biocomposites (left) were much lower than the TMV-bromide initiator (right), which indicates that the polymer was grafted to the surface of TMV by ATRP reaction. This result is due to the hydrophobic nature of HEMA in water. In comparison, the PEG modified PEGMA is higher than the layer of macro-initiator, due to the good water solubility of PEG. TEM images of the polymer modified TMV shows (FIG. 29d) that the diameter is much larger than the TMV macro-initiator (FIG. 29c).

### Example 2

In a typical experiment, an ethanol solution of P4VP was slowly added to a protein solution in 0.01 M phosphate buffer saline (PBS), pH=7.8, with stirring. Further details can be found in the experimental section. The mixtures were placed at room temperature to allow the ethanol to completely evaporate. Then the samples were observed by the naked eye to see whether they formed colloids (C) or aggregations (A), as shown in FIG. 1a. By checking the concentration of supernatant, A and S was formed from the complex of protein and P4VP.

Proteins with pIs higher than 9 such as Pap, Trp, RibA, Cyt, and Lys formed A, while proteins with pIs lower than 9 such as Pep, BSA, ChT, Fer, Hem, HSA, Str, Ova, Con A, Psm, Lip, Gel B, HRP, and Apo E4 formed C. It was previously reported that virus particles such as cowpea mosaic virus (CPMV) (pI 5.5, M_{w} 5600.0 kDa), turnip yellow mosaic virus (TYMV) (pI 3.8, M_{w} 5500.0 kDa), tobacco mosaic virus (TMV) (pI 3.5, M_{w} 5000.0 kDa), and bacteriophage M13 (pI 4.3, M_{w} 18000.0 kDa) can also formed C with P4VP. The results showed that pI, not MW, plays an important role in the assembly of proteins with P4VP. Furthermore, the composition of C and A is crucial for the study of the co-assembly process. The sample solution was first suspended and then the protein concentration of supernatant was evaluated. It was found that C was formed from the complex of protein and P4VP while A was a result of the precipitation of P4VP.

TABLE 2
ξ-potential and hydrodynamic size of BSA, BSA-P4VP (1-3), and
BSA-PCL-b-P2VP (1-3). BSA-P4VP (1-3) are the samples with mass
ratios of BSA to P4VP are 0.03, 0.14, and 0.29, respectively.
BSA-PCL-b-P2VP (1-3) are the samples with the mass ratios of BSA
to PCL-b-P2VP are 0.11, 0.29, and 0.57, respectively.

| | ξ-potential | Diameter |
|---|---|---|
| System | (mV) | (nm) |
| BSA | −35 ± 1 | N/A |
| BSA-P4VP 1 | −32 ± 1 | 331 ± 155 |
| BSA-P4VP 2 | −28 ± 1 | 214 ± 84 |
| BSA-P4VP 3 | −24 ± 1 | 170 ± 54 |
| BSA-PCL-b-P2VP 1 | −31 ± 1 | 260 ± 124 |
| BSA-PCL-b-P2VP 2 | −29 ± 2 | 221 ± 87 |
| BSA-PCL-b-P2VP 3 | −22 ± 1 | 186 ± 84 |

Different polymers with various physical properties were also used in our study. Hydrophobic polystyrene (PS), poly-y-benzyl-L-glutamate (PBLG), poly(lactic-co-glycolic acid) (PLGA) can form the A after added into the BSA solution. In addition, Nihydrin solution was used to confirm that the proteins is not a part of the A. More hydrophilic neutral polymers as poly(ethylen glycol) (PEG), poly(vinylalcohol) (PVA), poly(vinylpyrrolidone) (PVP), and charged polymer poly(ethyleneimine) (PEI), poly(sodium-4-styrenesulfonate) (PSS) do not form A and C, either. However, poly(3-(2-methoxyethoxy)methythiophenes) (PMT), polyaniline, P2VP, and PCL-b-P2VP can form the C with proteins. The hydrophobic dye such as pyrene and nile red can be encapsulated in the C, indicates that the structure of the C is similar to micelles. Hydrophobic-hydrophilic interactions can be considered as the major driving force for the formation of the structures. These results also show that with only hydrophobic or hydrophilic polymer is not enough to form the stable C. In addition, hydropobic polymers with certain functionalities, which can balance the interactions between themselves and proteins, can form the C with proteins.

In order to confirm our hypothesis, the co-assembly behaviors between proteins and P4VP were studied at various pH conditions. The surface charges of proteins can be modified by adjusting pHs. The assembly behaviors between proteins and P4VP were conducted at pHs of 5.5, 6.5, 7.8, 9.4, and 11.7, respectively. The proteins chosen were Pep (pI 2.8), TMV (pI 3.5), BSA (pI 4.8), Lip (pI 5.6), HRP (pI 7.2), ChT (pI 8.7), Pap (pI 9.6), and Rib A (pI 9.4). FIG. 2 shows the diagrams of proteins-P4VP at different pH values. HRP and ChT formed A with P4VP at pH 5.5 and 6.5 while forming C at pH 7.8, 9.4, and 11.7. This result can be attributed to ChT and HRP are positively charged at pH 5.5 and 6.5. Rib A and Pap formed C only at pH 11.7 while Pep, TMV, BSA, and Lip with P4VP formed C at various pHs because TMV, BSA, and Lip carried negative charges at all pHs while Rib A and Pap only had negative charges at pH 11.7. The results further confirmed our hypothesis that the electrostatic interactions are a major driving force for the formation of C. When the pH is below 3.2, the P4VP is protonated and becomes more hydrophilic (B), as shown in FIG. 2. When the pH is above 13, most proteins will be denatured (D). Therefore, based on this diagram, C can be easily prepared using different proteins under different buffer conditions.

Transmission electron microscopy (TEM), dynamic light scattering (DLS), and potential analysis were used to study the morphologies and physical properties of the C. Table 2 displays the ξ-potential and hydrodynamic size of BSA, BSA-P4VP (1-3), and BSA-PCL-b-P2VP (1-3). The size of protein-P4VP colloids can be readily adjusted by controlling the mass ratios of proteins and P4VP. DLS showed that the average diameters of BSA-P4VP (1-3) were 331, 214, and 170 nm, which were consistent with TEM results. The size of native BSA could not be detected due to the limitation of DLS. In addition, the average sizes of BSA-PCL-b-P2VP (1-3) were 260, 221, 186 nm, respectively. All the results showed that the sizes of colloids decreased while mass ratios of proteins to P4VP increased. Conclusively, a variety of proteins can act as stabilizers to stabilize the hydrophobic P4VP and form protein-P4VP colloids. The average sizes of ChT-P4VP (1-3) with the mass ratios of ChT to P4VP of 0.06, 0.13 and 0.25, were 558, 335, 263 nm, respectively. The proteins were considered to locate on the surface of P4VP balls. After BSA formed colloids with P4VP, ξ-potentials decreased from −35 to −32, −29, and −24 mV, as shown in Table 2. This results may be attributed to that partial BSA was attached to the surface of P4VP ball while the other part of BSA exposed to the solutions. Similar results were found for BSA-PCL-b-P2VP (1-3) samples ξ-potentials of BSA-PCL-b-P2VP (1-3) were −31, −29, −22 mV, respectively.

Circular dichroism (CD) experiments were performed to study the conformational structures of proteins. No CD signals representing P4VP particles were observed. As shown in FIG. 31, native ChT had two extreme minima of 204 nm and 227 nm, respectively. The CD spectra of ChT-P4VP 1 revealed a blue shift of one minimum valley from 204 to 203 nm, indicating that the conformational structure of the protein changed to a random coil and lost its native secondary structure. In addition, the characteristic minimum of the ChT spectrum at 227 nm still persisted, indicating that the protein retained its native secondary structure even after adsorbed onto P4VP balls. Since smaller size of particles of ChT/P4VP (2-3) with higher curvatures required more conformational change, more blue shifts were observed (FIG. 31). Similarly, native BSA (FIG. 31) had two extreme minima at 208 and 223 nm, respectively. Different from ChT-P4VP (1-3) samples, BSA-P4VP (1-3) samples still retained their conformational structures and no blue shifts were observed, indicating that the protein adsorptions had no effect on BSA integrity. Similar results can be observed for BSA-PCL-b-P2VP (1-3) samples. The difference between ChT and BSA could be explained by their different molecular weights and structures.

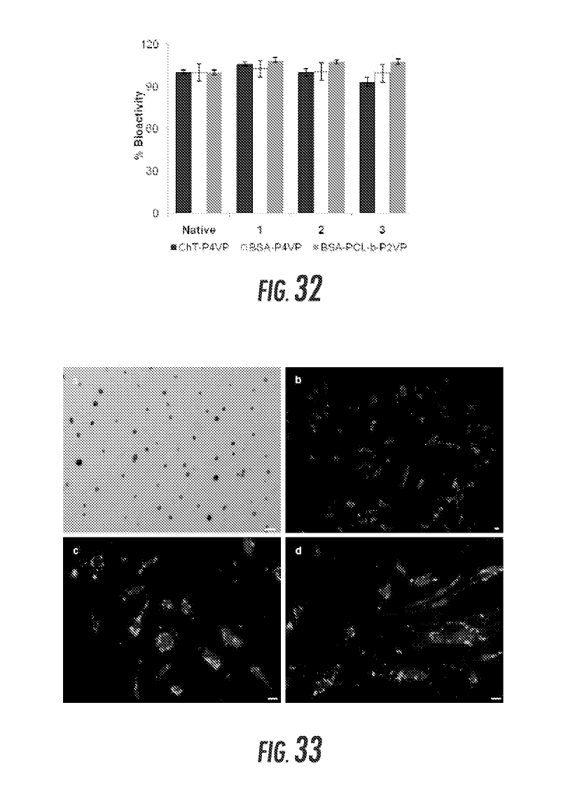

To probe the interactions between ChT and P4VP particles, the ChT-catalyzed hydrolysis of SPNA was examined. The activity of ChT in the presence of P4VP particles were normalized to that of free ChT. According to FIG. 32, ChT activities in ChT-P4VP (1-3) were comparable to native ChT. Bioactivity changes can be attributed to the conformational changes in ChT-P4VP samples. Similarly, BSA showed esterase-like activity toward aryl esters, such as p-nitrophenyl acetate, and its enzyme-like activity required conformational integrity of protein. The hydrolysis activity of p-nitrophenyl acetate via a native BSA along with the BSA-P4VP 1-3 was performed under the same conditions. As shown in FIG. 32, BSA-P4VP (1-3) and BSA-PCL-b-P2VP (1-3) activity was still comparable to native BSA, due to no conformational change.

ApoE is produced primarily in liver and brain. It is normally part of a lipoprotein complex, such as very-low-density lipoproteins (VLDL) and high-density lipoproteins (HDL). The presence of low-density lipoprotein receptors (LDLR) on the endothelial cells has long been demonstrated. These receptors can be utilized to transport drugs into the endothelial cells via targeted nanovectors. The cellular uptake properties of Apo E4-P4VP particles were checked with human umbilical-vein endothelial cells (HUVECs), as shown in FIG. 33. Apo E4 labeled with NHS-fluorescein was prepared and assembled with P4VP prior to the cell study. The size of the formed particle was between 100-200 nm. It was clearly observed that the particles are internalized by the HUVECs. Green fluorescence was observed for the particles. BSA and HSA labeled with NHS-fluorescein was prepared and assembled with P4VP and used as the control experiment. There are no fluorescence signals after the particles incubated with HUVECs for 24 h. These results also confirmed that the Apo E4 still remained its bioactivity after assembled with P4VP particles.

In summary, it was demonstrated that P4VP can assemble with proteins to form nanosized colloids which can be potentially applied to protein and drug delivery. The size of colloids can be easily controlled by adjusting the mass ratios of proteins and P4VP, confirmed by DLS and TEM. Besides, not only P4VP, but also biodegradable PCL-b-P2VP can be used to form colloids which will be a good vehicle for the application of drug delivery. In addition, the synthetic particles, composed of P4VP core with incorporated apoE4 protein on the surface, can be internalized by the HUVECs. This method may open up a new way to prepare the functional nanovectors with biodegradable polymer as the core and antibody as the shell, which can transport the therapeutic agents into specific part of the body.

### Experimental

Materials: Poly(4-vinylpyridine 60,000 Da), N-succinyl-L-phenylalanine p-nitroanilide (SPNA), p-nitrophenyl acetate, dimethylformamide (DMF), ethanol and tetrahydrofuran (THF), poly-y-benzyl-L-glutamate (PBLG) (Mw 150,000-300,000), poly(lactic-co-glycolic acid) (PLGA), poly(ethylene glycol) (PEG) (Mw 10,000), poly(vinyl alcohol) (PVA) (Mw 30,000-70,000), poly(2-vinylpyridine) (P2VP) (Mw 15,600), poly(sodium-4-styrenesulfonate) (PSS) (Mw 70,000), were purchased from Sigma-Aldrich company and used as received. Poly(ethyleneimine) (PEI) (Mw 70,000), poly(vinylpyrrolidone) (PVP) (Mw 1,300,000) were purchased from Alfa Aesar company. Polystyrene (PS) (Mw 24,600, PDI=1.2) was synthesized by RAFT polymerization. Poly(3-(2-methoxyethoxy)methythiophenes) (PMT) was synthesized with Mw 2,100. Water (18.2 MD) was obtained from Milli-Q system (Millipore). P2VP198-b-PCL310 (the subscripts indicate the block lengths; PDI=1.8) was purchased from Polymer Source Inc., Canada. Hemoglobin (Hem) (from human), Gelatin (Gel) (from bovine skin, Type B), α-chymotrypsinogen A (ChT A) (from bovine pancreas, Type II), Concanavalin A (Con A) (from Canavalia ensiformis (Jack bean), Type IV), a-chymotrypsin (ChT) (from bovine pancreas, Type II), Avidin (Avi) (from egg white), Ovabumin (Ova) (albumin from chicken egg white, Grade II), Lipase (Lip) (from candida rugosa), Papain (Pap) (from papaya latex), Myoglobin (Myo) (from horse heart, minimum 90% (PhastGel)), Ribonuclease A (Rib A) (from bovine pancreas), Trypsin (Try) (from bovine pancreas, Type I), Ferritin (Fer) (from horse spleen, Type I), and Mucin (Psm) (from porcine stomach, Type II) were purchased from Sigma-Aldrich company. Apo lipoprotein E (Apo E4) (from Human AG) was purchased from Fitzgerald Industries International. Bovine serum albumin fraction V (BSA) and Lysozyme (Lys) (from Hen Egg White) were purchased from Rockland company. Horseradish Peroxidase (HRP) and Strepavidin (Str) were purchased from Thermo scientific. Albumin from human serum (HSA) was purchased from Fluka.

Analysis: Circular dichroism (CD) was performed on a Jasco 815 spectrophotometer using a quartz cuvette with a 2 mm path length. Scans were taken from 180 to 250 nm at a rate of 100 nm/min, with a 1 nm step resolution and a 1 s response. Four scans were conducted at a constant temperature of 25° C., with a 10 min equilibration before the scans, and the average was reported. TEM analysis was performed by depositing 20 μL aliquots of each sample with a concentration between 0.1 and 0.5 mg mL-1 onto 100-mesh carbon coated copper grids for 2 min. The grids were then stained with 20 μL of uranyl acetate and observed with a Hitachi H-8000 electron microscope. The DLS analysis was performed by Submicron Particle Sizer AutodillutePAT Model 370. ξ-potential and light scattering measurements were performed on a Brookhaven Zeta PALS instrument. UV-vis absorption studies were performed using an Agilent 8453 UV-vis spectrometer.

A typical procedure to synthesize Protein-P4VP:

The experiments were performed in phosphate buffer saline (PBS buffer: 0.01 M, pH 7.8), unless specified. A solution of P4VP (Mw 60,000 Da) in ethanol (2.0 mg mL-1, 0.2 mL) was slowly added into a solution of proteins (0.4 mg) in PBS buffer with stirring. The final concentration of P4VP was 0.07 mg/mL, and the volume percentage of ethanol was ˜3%. The mixtures were placed at room temperature to allow ethanol to completely evaporate. Various characterizations of samples were conducted thereafter. Activity assay of ChT-P4VP and BSA-P4VP:

For activity test of ChT and ChT-P4VP samples, all the experiments were performed in sodium phosphate buffer solutions (5.0 mM, pH 7.4), unless specified. The functionality of ChT and the ChT-P4VP was determined by observing the absorbance associated with the hydrolysis product of SPNA in the presence of various sizes of the nanoparticles. At established time points, a SPNA stock solution in ethanol was added to the ChT-P4VP to reach a final [ChT] of 3.2 μM and [SPNA] of 2.0 mM. Activity assay was measured with the presence of ethanol by monitoring product formation for 30 min at 405 nm with a Molecular Device SPECTRAMax plus 384 with a microplate reader. The assays were performed multiple times, and the averages were reported. For activity assay of BSA and BSA-P4VP samples, all experiments were performed in potassium phosphate buffer (5.0 mM, pH 8.0), unless specified otherwise. The functionality of BSA and BSA-P4VP was examined by observing the absorbance of the hydrolysis of 4-nitrophynyl acetate in the presence of various sizes of the nanoparticles. A 10 μL solution of 10.0 mM 4-nitrophenyl acetate in acetonitrile was dissolved in 0.94 mL buffer solution. Then, the mixture was gently mixed with a BSA or BSA-P4VP (50 μL, [BSA]=0.27 mM). The mixture was allowed to incubate in the dark at room temperature for 30 min, measured at the absorbance of 405 nm to evaluate the activity. The activity assays were performed in multiple trails, and the averages were reported.

Cell Culture Human umbilical-vein endothelial cells (HUVECs) were maintained in F12K media supplemented with 10% fetal bovine serum (FBS), penicillin-streptomycin-Fungizone (PSF), heparin, and endothelial cell growths (EGGS) at 37° C. in 5% CO2. HUVECs were used at passage 8-9. A confluent 25 cm2 flask of cells was dispersed using trypsin/EDTA solution (brand). Cells were resuspended in media. Approximately 10,000 cells were added to each well of 6-well tissue culture plates (brand) and were allowed to adhere for 48 h.

Cellular association with and localization of fluorescently labeled protein-polymer in HUVEC was assessed by confocal laser scanning microscopy using a Leica TCS-SP system. Cells were incubated for 2 and 24 h with the different controls. After incubation, cells were washed with PBS and fixed with 4% formaldehyde before visualization. Microscopy settings were identical for the different control to allow comparison of the results.

### Example 3

### Formation of CPMV-PS Particles Using Polystyrene (PS) End Capped with Free Amine Groups

CPMV-PS particles were obtained by mixing PS beads (˜130 um) with CPMV particles in PBS buffer with pH 7.8. PS beads coated with PEG-NH₂ groups are positively charged in aqueous solutions. CPMV is negatively charged because the isoelectric point (pI) of CPMV is around 5.5. Therefore, the driving force of formation of CPMV-PS is primarily based on the strong electrostatic interactions. Wide-type CPMV was labeled covalently with the N,N,N/,N/-tetramethylrhodamine NHS ester fluorescent rhodamine dye to form CPMV-Rh. UV-Vis and fast protein liquid chromatography (FPLC) showed that the rhodamine was conjugated to the surface of CPMV and the CPMV particles were still intact after the modification. The CPMV-P4VP particles showed strong fluorescent signals, indicating the rhodamine labeled CPMV particles were coated on the PS beads. As a control experiment, there was no fluorescence signal for the PS particles. The cross section analysis of confocal microscopy showed that a ring-like morphology was formed, indicating the CPMVs were only coated on the surface of PS.

Other BNP-PS particles assembled using different BNPs such as spherical P22 and rodlike TMV were also formed. Bacteriophage P22 is another spherical virus with a diameter ˜60 nm in diameter with a very short tail. A native TMV particle has 2130 identical protein subunits arranged helically around the genomic singlestrand RNA with a 300 nm in length and 18 nm in diameter. It was clearly observed that CPMV, P22, and TMV all formed long-range ordered structures on the surface of PS by FESEM imaging. In addition, different sizes of these core-shell structures can be fabricated with PS with different diameters including 10 μm and 320 μm. Ferritin (Fe), another protein cage with a diameter between 12-14 nm, formed the PS-Fe colloids, which was confirmed by EDX. However, due to the limitation of FESEM, it was difficult to observe the ferritin particles by FESEM.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A bionanocomposite comprising
a core comprising a polymer having pyridine functional groups, wherein the core defines an outer surface having a surface area; and
a shell comprising a plurality of biomacromolecules, wherein the shell is positioned about the outer surface of the core such that the shell covers at least about 50% of the surface area of the polymeric core;
wherein the polymer having pyridine functional groups non-covalently interacts with the biomacromolecules to form the bionanocomposite.

2. The bionanocomposite as in claim 1, wherein the biomacromolecules comprise virus particles.

3. The bionanocomposite as in claim 2, wherein the virus particles comprise cowpea mosaic virus, turnip mosaic virus, tobacco mosaic virus, bacteriophage M13, or combinations thereof.

4. The bionanocomposite as in claim 1, wherein the biomacromolecules comprise proteins.

5. The bionanocomposite as in claim 4, wherein the proteins have an isoelectric point lower than about 9.

6. The bionanocomposite as in claim 1, wherein the core further comprises a biologically active material.

7. The bionanocomposite as in claim 1, wherein the bionanocomposite has a diameter from about 10 nanometers to about 200 nanometers.

8. The bionanocomposite as in claim 1, wherein the polymer having pyridine functional groups comprises poly(4-vinylpyridine), poly(2-vinyl pyridine), or copolymers or combinations thereof.

9. The bionanocomposite as in claim 8, wherein the polymer having pyridine functional groups comprises poly(styrene-b-4-vinyl pyridine), poly(styrene-b-2-vinyl pyridine), poly(2-vinyl pyridine-b-c-caprolactone), poly(ethylene oxide-b-4-vinyl pyridine), poly(styrene-b-4-vinylpyridine-b-styrene), poly(4-vinyl pyridine-b-styrene-b-4-vinyl pyridine), poly(styrene-b-4-vinyl pyridine-b-ethylene oxide) or combinations thereof.

10. The bionanocomposite as in claim 1, wherein the shell is configured to disassociate from the core when in an aqueous solution having a pH of less than about 3.

11. A method of forming a bionanocomposite defining a shell and a core, the method comprising
non-covalently attaching biomacromolecules about a polymeric core, wherein the polymeric core comprises a polymer having pyridine functional groups and defines an outer surface having a surface area, and wherein the biomacromolecules cover at least about at least about 50% of the surface area of the polymeric core to form a shell.

12. The method as in claim 11, wherein the biomacromolecules are attached to the polymeric core by
combining an organic solution containing the polymer in an organic solvent with an aqueous solution containing the biomacromolecules to form an emulsion;
mixing the emulsion; and
removing the organic solvent.

13. The method as in claim 12, wherein the organic solvent is removed by dialysis or evaporating at room temperature.

14. The method as in claim 12, wherein the organic solution is simultaneously dripped into and mixed with the aqueous solution.

15. The method as in claim 11, wherein the biomacromolecules comprise virus particles.

16. The method as in claim 15, wherein the virus particles comprise cowpea mosaic virus, turnip mosaic virus, tobacco mosaic virus, bacteriophage M13 or combinations thereof.

17. The method as in claim 11, wherein the biomacromolecules comprise proteins.

18. The method as in claim 17, wherein the proteins have an isoelectric point lower than about 9.

19. The method as in claim 1, wherein the core further comprises a biologically active material.

20. The method as in claim 1, wherein the polymer having pyridine functional groups comprises poly(4-vinylpyridine), poly(2-vinylpyridine), or copolymers or combinations thereof.

21. The method as in claim 20, wherein the polymer having pyridine functional groups comprises poly(styrene-b-4-vinyl pyridine), poly(styrene-b-2-vinyl pyridine), poly(2-vinyl pyridine-b-ε-caprolactone), poly(ethylene oxide-b-4-vinyl pyridine), poly(styrene-b-4-vinylpyridine-b-styrene), poly(4-vinyl pyridine-b-styrene-b-4-vinyl pyridine), poly(styrene-b-4-vinyl pyridine-b-ethylene oxide) or combinations thereof.
